(19) United States
(12) Patent Application Publication
(10) Pub. No.: US 2023/0223100 A1
(43) Pub. Date: Jul. 13, 2023

(54) INTER-MODEL PREDICTION SCORE RECALIBRATION

(71) Applicants: Illumina, Inc., San Diego, CA (US); Illumina Cambridge Limited, Cambridge (GB)

(72) Inventors: Tobias HAMP, Cambridge (GB); Kai-How FARH, Hillsborough, CA (US)

(73) Assignees: Illumina, Inc., San Diego, CA (US); Illumina Cambridge Limited, Cambridge (GB)

(21) Appl. No.: 17/947,053

(22) Filed: Sep. 16, 2022

Related U.S. Application Data
(60) Provisional application No. 63/294,830, filed on Dec. 29, 2021.
(60) Provisional application No. 63/294,828, filed on Dec. 29, 2021.
(60) Provisional application No. 63/294,827, filed on Dec. 29, 2021.
(60) Provisional application No. 63/294,820, filed on Dec. 29, 2021.
(60) Provisional application No. 63/294,816, filed on Dec. 29, 2021.
(60) Provisional application No. 63/294,813, filed on Dec. 29, 2021.

(51) Int. Cl. G16B 5/20; G16B 20/20; G16B 40/00

(52) U.S. Cl. CPC G16B 5/20; G16B 20/20; G16B 40/00

(57) ABSTRACT

The technology disclosed relates to inter-model prediction score recalibration. In one implementation, the technology disclosed relates to a system including a first model that generates, based on evolutionary conservation summary statistics of amino acids in a target protein sequence, a first pathogenicity score-to-rank mapping for a set of variants in the target protein sequence; and a second model that generates, based on epistasis expressed by amino acid patterns spanning the target protein sequence and a plurality of non-target protein sequences aligned in multiple sequence alignment, a second pathogenicity score-to-rank mapping for the set of variants. The system also includes a reassignment logic that reassigns pathogenicity scores from the first set of pathogenicity scores to the set of variants based on the first and second score-to-rank mappings, and an output logic to generate a ranking of the set of variants based on the reassigned scores.

### PRIORITY APPLICATIONS

This application claims priority to or the benefit of U.S. Provisional Pat. Application No. 63/294,828, titled, “Inter-Model Prediction Score Recalibration,” by Tobias HAMP and Kai-How FARH, filed Dec. 29, 2021 (Attorney Docket No. ILLM 1067-⅟IP-2301-PRV); U.S. Provisional Pat. Application No. 63/294,813, titled, “Periodic Mask Pattern for Revelation Language Models,” by Tobias HAMP, Anastasia Susanna Dagmar DIETRICH, Yibing WU, Jeffrey Mark EDE, Kai-How FARH, filed Dec. 29, 2021 (Attorney Docket No. ILLM 1063-⅟IP-2296-PRV); U.S. Provisional Pat. Application No. 63/294,816, titled, “Classifying Millions of Variants of Uncertain Significance Using Primate Sequencing And Deep Learning,” by Hong GAO, Tobias HAMP, Joshua Goodwin Jon MCMASTER-SCHRAIBER, Laksshman SUNDARAM, Kai-How FARH, filed Dec. 29, 2021 (Attorney Docket No. ILLM 1064-⅟IP-2297-PRV); U.S. Provisional Pat. Application No. 63/294,820, titled, “Identifying Genes With Differential Selective Constraint Between Humans And Non-human Primates,” by Hong GAO, Joshua Goodwin Jon MCMASTER-SCHRAIBER, Kai-How FARH, filed Dec. 29, 2021 (Attorney Docket No. ILLM 1065-⅟IP-2298-PRV); U.S. Provisional Pat. Application No. 63/294,827, titled, “Deep Learning Network for Evolutionary Conservation,” by Sabrina RASHID and Kai-How FARH, filed Dec. 29, 2021 (Attorney Docket No. ILLM 1066-⅟IP-2299-PRV); U.S. Provisional Pat. Application No. 63/294,830, titled, “Species-differentiable Evolutionary Profiles,” by Tobias HAMP and Kai-How FARH, filed Dec. 29, 2021 (Attorney Docket No. ILLM 1068-⅟IP-2302-PRV). The provisional applications are hereby incorporated by reference for all purposes.

### INCORPORATIONS BY REFERENCE

The following are incorporated by reference for all purposes as if fully set forth herein, and should be considered part of, this provisional patent filing:

U.S. Pat. Application titled, “INTER-MODEL PREDICTION SCORE RECALIBRATION DURING TRAINING,” filed contemporaneously (Attorney Docket No. ILLM 1067-3/IP-2301-US2);

Sundaram, L. et al. Predicting the clinical impact of human mutation with deep neural networks. Nat. Genet. 50, 1161-1170 (2018) (referred to herein as “PrimateAI”);

Jaganathan, K. et al. Predicting splicing from primary sequence with deep learning. Cell 176, 535-548 (2019) (referred to herein as “SpliceAI”);

U.S. Pat. Application No. 62/573,144, titled “TRAINING A DEEP PATHOGENICITY CLASSIFIER USING LARGE-SCALE BENIGN TRAINING DATA,” filed Oct. 16, 2017 (Attorney Docket No. ILLM 1000-⅟IP-1611-PRV) (referred to herein as “PrimateAI”);

US. Pat. Application No. 62/573,149, titled “PATHOGENICITY CLASSIFIER BASED ON DEEP CONVOLUTIONAL NEURAL NETWORKS (CNNs),” filed Oct. 16, 2017 (Attorney Docket No. ILLM 1000-2/IP-1612-PRV) (referred to herein as “PrimateAI”);

U.S. Pat. Application No. 62/573,153, titled “DEEP SEMI-SUPERVISED LEARNING THAT GENERATES LARGE-SCALE PATHOGENIC TRAINING DATA,” filed Oct. 16, 2017 (Attorney Docket No. ILLM 1000-3/IP-1613-PRV) (referred to herein as “PrimateAI”);

U.S. Pat. Application No. 62/582,898, titled “PATHOGENICITY CLASSIFICATION OF GENOMIC DATA USING DEEP CONVOLUTIONAL NEURAL NETWORKS (CNNs),” filed Nov. 7, 2017 (Attorney Docket No. ILLM 1000-4/IP-1618-PRV) (referred to herein as “PrimateAI”);

U.S. Pat. Application No. 16/160,903, titled “DEEP LEARNING-BASED TECHNIQUES FOR TRAINING DEEP CONVOLUTIONAL NEURAL NETWORKS,” filed on Oct. 15, 2018 (Attorney Docket No. ILLM 1000-5/IP-1611-US) (referred to herein as “PrimateAI”);

U.S. Pat. Application No. 16/160,986, titled “DEEP CONVOLUTIONAL NEURAL NETWORKS FOR VARIANT CLASSIFICATION,” filed on Oct. 15, 2018 (Attorney Docket No. ILLM 1000-6/IP-1612-US) (referred to herein as “PrimateAI”);

U.S. Pat. Application No. 16/160,968, titled “SEMI-SUPERVISED LEARNING FOR TRAINING AN ENSEMBLE OF DEEP CONVOLUTIONAL NEURAL NETWORKS,” filed on Oct. 15, 2018 (Attorney Docket No. ILLM 1000-7/IP-1613-US) (referred to herein as “PrimateAI”);

U.S. Pat. Application No. 16/160,978, titled “DEEP LEARNING-BASED SPLICE SITE CLASSIFICATION,” filed on Oct. 15, 2018 (Attorney Docket No. ILLM 1001-4/IP-1610-US) (referred to herein as “SpliceAI”);

U.S. Pat. Application No. 16/407,149, titled “DEEP LEARNING-BASED TECHNIQUES FOR PRE-TRAINING DEEP CONVOLUTIONAL NEURAL NETWORKS,” filed May 8, 2019 (Attorney Docket No. ILLM 1010-⅟IP-1734-US) (referred to herein as “PrimateAI”);

U.S. Pat. Application No. 17/232,056, titled “DEEP CONVOLUTIONAL NEURAL NETWORKS TO PREDICT VARIANT PATHOGENICITY USING THREE-DIMENSIONAL (3D) PROTEIN STRUCTURES,” filed on Apr. 15, 2021, (Atty. Docket No. ILLM 1037-2/IP-2051-US) (referred to herein as “PrimateAI 3D”);

U.S. Pat. Application No. 63/175,495, titled “MULTI-CHANNEL PROTEIN VOXELIZATION TO PREDICT VARIANT PATHOGENICITY USING DEEP CONVOLUTIONAL NEURAL NETWORKS,” filed on Apr. 15, 2021, (Atty. Docket No. ILLM 1047-⅟IP-2142-PRV) (referred to herein as “PrimateAI 3D”);

U.S. Pat. Application No. 63/175,767, titled “EFFICIENT VOXELIZATION FOR DEEP LEARNING,” filed on Apr. 16, 2021, (Atty. Docket No. ILLM 1048-⅟IP-2143-PRV) (referred to herein as “PrimateAI 3D”);

U.S. Pat. Application No. 17/468,411, titled “ARTIFICIAL INTELLIGENCE-BASED ANALYSIS OF PROTEIN THREE-DIMENSIONAL (3D) STRUCTURES,” filed on Sep. 7, 2021, (Atty. Docket No. ILLM 1037-3/IP-2051A-US) (referred to herein as “PrimateAI 3D”);

U.S. Provisional Pat. Application No.: 63/253,122, titled “PROTEIN STRUCTURE-BASED PROTEIN LANGUAGE MODELS,” filed Oct. 6, 2021 (Attorney Docket No. ILLM 1050-⅟IP-2164-PRV) (referred to herein as “JigsawAI”);

U.S. Provisional Pat. Application No.: 63/281,579, titled “PREDICTING VARIANT PATHOGENICITY FROM EVOLUTIONARY CONSERVATION USING THREE-DIMENSIONAL (3D) PROTEIN STRUCTURE VOXELS,” filed Nov. 19, 2021 (Attorney Docket No. ILLM 1060-⅟IP-2270-PRV) (referred to herein as “JigsawAI”); and

U.S. Provisional Pat. Application No.: 63/281,592, titled “COMBINED AND TRANSFER LEARNING OF A VARIANT PATHOGENICITY PREDICTOR USING GAPED AND NON-GAPED PROTEIN SAMPLES,” filed Nov. 19, 2021 (Attorney Docket No. ILLM 1061-⅟IP-2271-PRV) (referred to herein as “JigsawAI”).

### FIELD OF THE TECHNOLOGY DISCLOSED

The technology disclosed relates to artificial intelligence type computers and digital data processing systems and corresponding data processing methods and products for emulation of intelligence (i.e., knowledge-based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using deep neural networks to analyze ordered data.

### BACKGROUND

Protein analysis often presents a so-called small data problem, in contrast to natural language processing. The size of the Internet, including digitized libraries, presents a virtually unlimited data set for training natural language models of all types. In contrast, fewer than 1000 species, at the time of this application, have reliably sequenced full proteomes. Compounding the small data problem, researchers have found that protein-by-protein analysis has advantages over trying to generalize across a whole proteome. In contrast to the linear structure of natural language, which progresses in words and sentences from the top to the bottom of a text, proteins undergo three-dimensional folding of the amino acid sequence, described in tertiary and even quaternary levels of the organization.

Protein synthesis begins with genes. Each human has a unique genetic code, though a large portion of the human genetic code is common for all humans. DNA code is transcribed into RNA, which then translates into a chain of amino acids followed by three-dimension folding of the sequence to form the final protein. In some cases, a human genetic code may include an outlier, called a genetic variant, that may be common among individuals of a relatively small group of the human population. The genetic variant may affect the translation of amino acids, which causes an amino acid variant. For example, a particular human protein may comprise a specific sequence of amino acids, whereas a variant of that protein may differ by one amino acid in the otherwise same specific sequence. Genetic variants may be pathogenetic, leading to diseases. Though most of such genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein. Consider a protein, its amino acid sequence determines its structure and function. When an amino acid variant replaces its original amino acid in the sequence, it may change the structure (e.g., protein folding), biochemical function, and evolvability of the protein.

It is widely recognized that further developments are needed to encode biological information and to encapsulate the complexity of the sequence space, structural space, and functional relationships of biomolecules and biomolecular systems. See, e.g., Bepler, T., & Berger, B.Learning the protein language: Evolution, structure, and function. Cell Systems, 2019, 12(6), 654-669. In 2020, Facebook’s AI Laboratory produced a transformer model, DeepSequence that it trained on 250 million protein sequences. See, e.g., Rives, A., Meier, J., Sercu, T., Goyal, S., Lin, Z., Liu, J., Guo D., Ott M., Zitnick, C.L., Ma, J., Fergus, R. Biological structure and function emerge from scaling unsupervised learning to 250 million protein sequences.Proceedings of the National Academy of Sciences of the United States of America, 2021, 118(15). But as Bepler & Berger explained, much work remains to refine and combine such divergent approaches to protein analysis.

For example, an important question in molecular evolution is whether an amino acid that occurs at a given site makes an independent contribution to fitness, or whether its contribution depends on the state of other sites in the organism’s genome. The phenotypic effect of a mutation at one genetic site often depends on alleles at other sites, a phenomenon known as epistasis. As a result, any mutation is expected to be contingent on earlier mutations and the fate of this mutation depends on the evolutionary history of the population. Therefore, epistasis can profoundly influence the process of evolution in populations and shape the patterns of divergence across species. Understanding the nature of epistatic interactions between sites will allow us to address basic questions in biology at the molecular scale, such as how large a role history plays in evolution, whether later events depend critically on specific earlier events, or do all events occur more or less independently.

The central properties of proteins—folding, biochemical function, and evolvability—arise from a global pattern of cooperative energetic interactions between amino acid residues. When introducing amino acid substitutions in a protein, cooperativity manifests itself as context-dependence of the effects of those mutations, or epistasis. Knowledge of the extent and distribution of epistasis in a protein is essential for understanding its evolution. For example, when a certain functional improvement requires a combination of mutations that are individually unfavorable, no single-mutation trajectory exists that increases fitness at each step, and evolution towards the new functionality will be hampered. Being able to uncover epistasis is relevant for the reconstruction of phylogenetic trees and for estimating the evolutionary potential of antibiotic resistance genes, and viruses, but also for protein engineering efforts that make use of directed evolution: information on epistatic architectures should prove useful in the selection of evolvable templates in focusing mutations to highly-epistatic regions of a protein, or in identifying cooperative units for DNA shuffling experiments. Epistasis can be co-evolutionary, where epistatic interactions lead to co-evolution among the loci.

Models that can predict molecular phenotypes directly from biological sequences can be used as in silico perturbation tools to probe the associations between genetic variation and phenotypic variation and have emerged as new methods for quantitative trait loci identification and variant prioritization. These approaches are of major importance given that the majority of variants identified by genome-wide association studies of complex phenotypes are non-coding, which makes it challenging to estimate their effects and contribution to phenotypes. Moreover, linkage disequilibrium results in blocks of variants being co-inherited, which creates difficulties in pinpointing individual causal variants. Thus, sequence-based deep learning models that can be used as interrogation tools for assessing the impact of such variants offer a promising approach to finding potential drivers of complex phenotypes. More importantly, an opportunity rises to use deep learning models, not only for predicting variant pathogenicity for a single variant introduced at a given location of the protein sequence but also considering context-dependence of the effects of the variant, in other words, epistasis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The color drawings also may be available in PAIR via the Supplemental Content tab. In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which:

FIG. 1 illustrates an example system including two models for generating pathogenicity score-to-rank mappings for target variants and recalibrating pathogenicity scores of the variants, in accordance with one implementation of the technology disclosed.

FIG. 2 illustrates an example of the first model using different types of input to a pathogenicity classifier for variant pathogenicity prediction, in accordance with one implementation of the technology disclosed.

FIG. 3 illustrates an example of a position-specific frequency matrix (PSFM).

FIG. 4 illustrates an example of an input position-specific scoring matrix (PSSM).

FIG. 5 illustrates an example of multiple sequence alignment, in accordance with one implementation of the technology disclosed.

FIG. 6 is a schematic representation of an encoder-decoder architecture, in accordance with one implementation of the technology disclosed.

FIG. 7 shows an overview of an attention mechanism added onto an RNN encoder-decoder architecture, in accordance with one implementation of the technology disclosed.

FIG. 8 is a schematic representation of the calculation of self-attention showing one attention head), in accordance with one implementation of the technology disclosed.

FIG. 9 depicts several attention heads in a Transformer block, in accordance with one implementation of the technology disclosed.

FIG. 10 shows parallel execution of multi-head attention logics, in accordance with one implementation of the technology disclosed.

FIG. 11 portrays one encoder layer of a Transformer network, in accordance with one implementation of the technology disclosed.

FIG. 12 shows a schematic overview of a Transformer model, in accordance with one implementation of the technology disclosed.

FIG. 13A shows a Vision Transformer (ViT), in accordance with one implementation of the technology disclosed.

FIG. 13B shows a Transformer block used by the Vision Transformer, in accordance with one implementation of the technology disclosed.

FIGS. 14A, 14B, 14C, and 14D show details of the Transformer block of FIG. 13B, in accordance with one implementation of the technology disclosed.

FIG. 15 shows an example source code implementing the Vision Transformer.

FIG. 16 illustrates an example of the second model using multiple sequence alignment (MSA) as input to a generative model for variant pathogenicity prediction, in accordance with one implementation of the technology disclosed.

FIG. 17 illustrates an example of inter-model variant pathogenicity score reassignment, in accordance with one implementation of the technology disclosed.

FIG. 18 illustrates another example of inter-model variant pathogenicity score reassignment, in accordance with one implementation of the technology disclosed.

FIG. 19 illustrates an example system including two models for generating pathogenicity score-to-rank mappings for target variants and rescaling pathogenicity scores, in accordance with one implementation of the technology disclosed.

FIG. 20 illustrates variant pathogenicity prediction results of rare variants from UK Biobank and rare variants from patients with developmental delay using an inter-model variant pathogenicity score recalibration system, in accordance with one implementation of the technology disclosed.

FIG. 21 is an example computer system that can be used to implement the technology disclosed.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general-purpose signal processor or a block of random-access memory, hard disk, or the like) or multiple pieces of hardware. Similarly, the programs may be stand-alone programs, may be incorporated as subroutines in an operating system, may function in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel, or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between.

Logic as used herein (e.g., reassignment logic, rescaling logic, combination logic), can be rule-based and implemented in the form of a computer product including a non-transitory computer readable storage medium with computer usable program code for performing the method steps described herein. The “logic” can be implemented in the form of an apparatus including a memory and at least one processor that is coupled to the memory and operative to perform exemplary method steps. The rule-based reassignment and rescaling logics can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media). In one implementation, the logic implements a data processing function. The logic can be a general purpose, single core or multicore, processor with a computer program specifying the function, a digital signal processor with a computer program, configurable logic such as an FPGA with a configuration file, a special purpose circuit such as a state machine, or any combination of these. Also, a computer program product can embody the computer program and configuration file portions of the logic.

This application uses the terms “recalibration”, “reassignment” and “re-ranking” interchangeably.

### System for Inter-model Variant Pathogenicity Score Reassignment

Genetic variants can be pathogenetic, leading to diseases. Though most genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein.

The technologies disclosed can be used to improve the quality of pathogenic variant identification. Our models output probabilities for protein missense variants. It can be applied to score all possible variants at every position in most human proteins, achieving high performance on our pathogenicity prediction benchmarks. Our models are applicable to proteins from any species, and so are useful for protein engineering. In addition, intermediate model features can be used for protein structure prediction. The model performance smoothly increases with model size and compute, so other researchers will benefit from transfer learning from a large model trained with more compute.

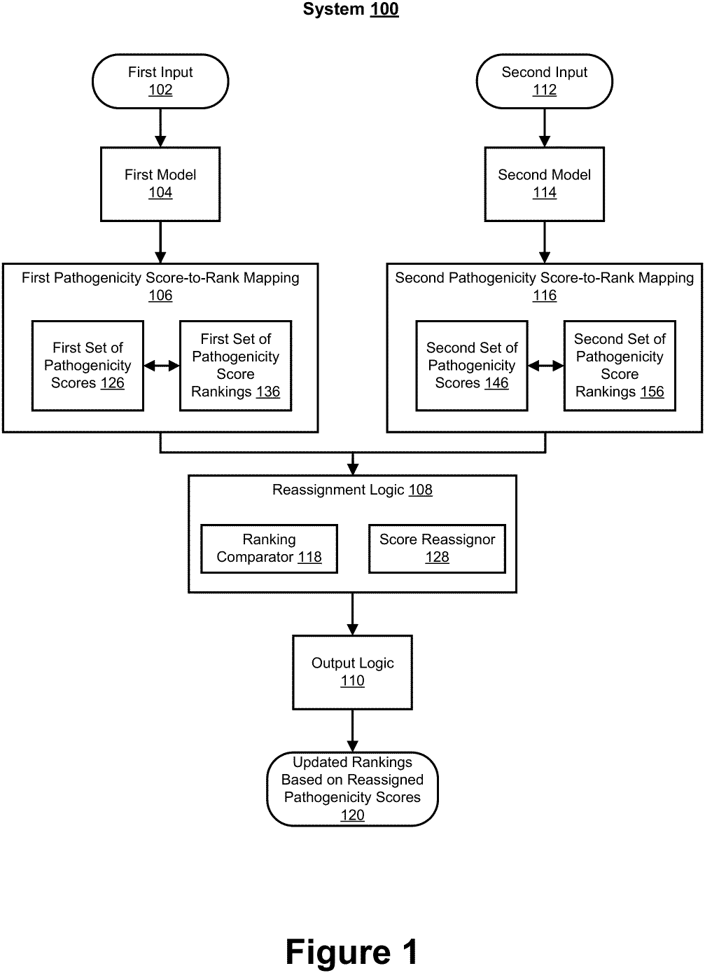

FIG. 1 illustrates an example system including two models for generating variant pathogenicity score-to-rank mappings and recalibrating the pathogenicity score of the variants. The system 100 includes two models, each independently generating a pathogenicity score-to-rank mapping for a set of target variants. The first model 104, based on the first input 102, generates a first pathogenicity score-to-rank mapping including a first set of pathogenicity scores 126 mapped to a first set of pathogenicity score rankings 136.

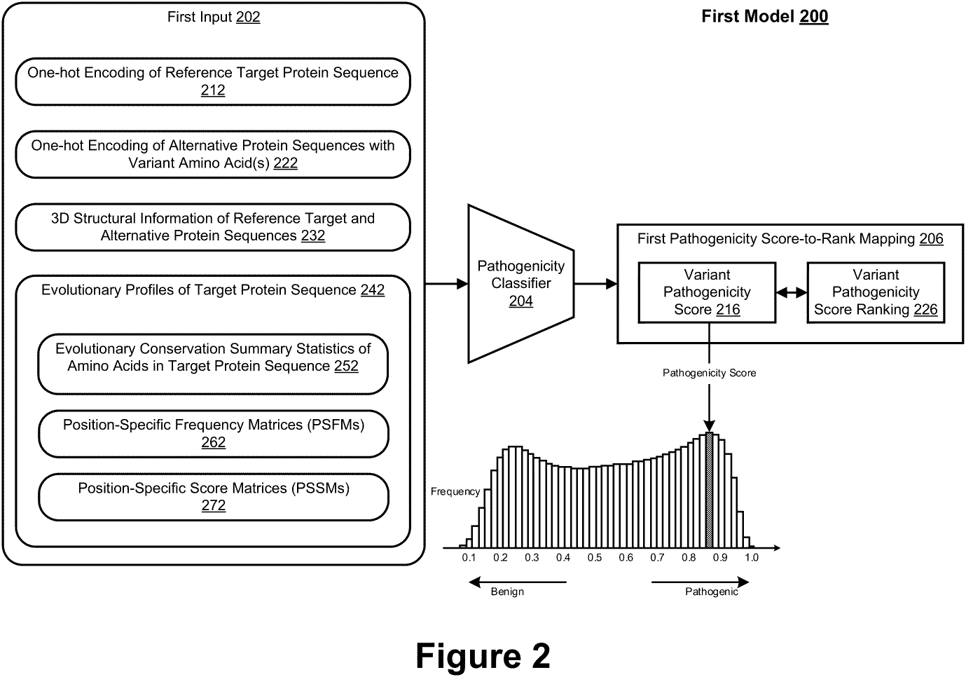

In one implementation, the first put 102 includes sequence information, structural information, and evolutionary profiles of a reference target protein sequence. Sequence information may include amino acid sequences of the reference target protein sequence and non-target protein sequences aligned with the reference target protein sequence in a multiple sequence alignment. Structural information may represent the three-dimensional (3D) structure of the reference target protein sequence due to the interactions between amino acids that make up the protein. Structural information may also represent locally folded structures that form within the protein due to interactions between atoms of the backbone chain (e.g., α-helix, β-sheet). When the reference target protein sequence includes multiple polypeptide chains known as subunits, structural information may also represent the quaternary structure of the protein, i.e., the association of subunits into a closely packed arrangement. Evolutionary profiles may include evolutionary conservation summary statistics of amino acids in the target protein sequence. Evolutionary conservation refers to the presence of similar genes, portions of genes, or chromosome segments in different species, reflecting both the common origin of species and important functional property of the conserved elements. The first input 102 may also include an alternative protein sequence with one or more amino acid variants substituting the original amino acids at certain locations of the reference target protein sequence. Different types of the first input 102 will be described in more detail in FIG. 2.

The first model 104 can be a site-independent model that factorizes single-position variations in a plurality of aligned sequences. In other words, the first model 104 predicts the pathogenicity score of a single variant located in a particular location of the protein sequence without considering the context-dependence of the effects of the variant. In another implementation, the first model 104 can be a pairwise-interaction model that factorizes two-position variations in a plurality of aligned sequences. The incorporation of inter-site dependencies in the pairwise interaction model can lead to a more accurate prediction of the effects of the variant.

In one implementation, the first set of pathogenicity scores 126 generated from the first model 104 has a score scale, for example, ranging from 0 to 1, to differentiate benign variants from pathogenic ones. That is, a lower score indicates a benign variant mapped to a higher ranking while a higher score indicates a pathogenic variant mapped to a lower ranking.

The second model 114, based on the second input 112, generates a second pathogenicity score-to-rank mapping 116 that includes a second set of pathogenicity scores 146 mapped to a second set of pathogenicity score rankings 156.

In one implementation, the second input 112 includes sequence information of a target protein sequence from a target species and a plurality of non-target protein sequences aligned with the target protein sequence in a multiple sequence alignment (MSA). MSA is a sequence alignment of multiple homologous protein sequences to a target protein. It is an important step in comparative analyses and property prediction of biological sequences since a lot of information, for example, evolution and coevolution clusters, are generated from the MSA and can be mapped to the target sequence of choice or on the protein structure. Compared to pairwise alignments, MSA provides more information since they show conserved regions within a protein family which are of structural and functional importance. MSA used as the second input 112 can be a collection of tens to thousands of aligned protein sequences expanding from a target species (e.g., human) to a variety of homologous non-target species (e.g., primate, mammals, vertebrate). The second model 114 can distinguish between proteins that belong to the same protein family defined by MSA from those that do not. Proteins with pathogenic variants are predicted as outside of the protein family and those with benign variants as inside of the family.

In one implementation, the second set of pathogenicity scores 146 generated from the second model 114 also has a score scale, for example, ranging from a maximum real number represented digitally to a minimum real number represented digitally, to differentiate pathogenic variants from benign variants. That is, each score may range anywhere from -∞ to +∞, where a lower score indicates a pathogenic variant mapped to a lower-ranking while a higher score indicates a benign variant mapped to a higher ranking. Yet in other implementations, a lower score may indicate a benign variant mapped to a higher ranking and a higher score indicates a pathogenic variant mapped to a lower ranking. The second set of pathogenicity scores can also be re-scaled to a preset scale, in order to compare with the first set of pathogenicity scores.

A person skilled in the art will appreciate that the present disclosure does not intend to limit the scales of the pathogenicity scores generated from the two models, or the correlation between the numerical values of pathogenicity scores and corresponding score rankings.

A person skilled in the art will also appreciate that the present disclosure does not intend to limit the types of inputs that are fed into the two models or the architectures of the models. The first model 104 and the second model 114 may have different architectures while the first input 102 and the second input 112 are substantially similar. Alternatively, the first model 104 and the second model 114 may have substantially similar architectures while the first input 102 and the second input 112 are different. In one implementation, the first input 102 used to feed the first model 104 may be distinct from the second input 112 used for the second model 114, and because of this input distinction, for the same set of variants in a target protein sequence, the two models may generate different outputs of pathogenicity scores and corresponding rankings. In another implementation, the first model 104 may have a different architecture from the second model 114, and because of this distinction, for the same set of variants in a target protein sequence, the two models may generate different outputs of pathogenicity scores and corresponding rankings.

As illustrated in FIG. 1, the system 100 further includes a reassignment logic 108 including a ranking comparator 118 and a score reassignor 128. In one implementation, for the same variant in a target protein sequence, the ranking comparator 118 compares its corresponding ranking generated from the first model 104 with the ranking generated from the second model 114. In view of the comparison, the score reassignor 128, reassigns a pathogenicity score to this variant. In one implementation, for a set of variants, the score reassignor 128 maintains the value and scale of each score within the first set of pathogenicity scores 126, but re-assigns these scores to the set of variants based on the second score-to-rank mapping 116. For example, variant A has a given pathogenicity score of 0.5 mapped to a corresponding ranking of 3, both generated from the first model 104. Meanwhile, variant A has a different pathogenicity score and a corresponding ranking of 2, both generated from the second model 114. The ranking comparator 118 compares the two rankings, namely, 3 and 2. The score reassignor 128, based on the ranking of 2 generated from the second model 114, re-assigns a given score of 0.3 to variant A, where the score of 0.3 was originally assigned to another variant B that had a ranking of 2 generated from the first model 104. Accordingly, output logic 110 generates an updated ranking 120 based on the reassigned pathogenicity score to each variant. The updated ranking 120 can be different from the first set of pathogenicity score rankings 136. For example, the updated ranking of variant A is 2, different from its original ranking of 3 generated from the first model 104.

In some implementations, variants occur in different residues (e.g., microenvironmental context) within a protein sequence. Accordingly, the reassignment logic 108 (including the ranking comparator 118 and the score reassignor 128) automatically compares and assigns the pathogenicity scores to variants within each residue and across different residues. As an example, variants A, B, C and D, referring to respective variant amino acids, are present at two different residues in a protein sequence, namely, Residue 1 and Residue 2. The ranking comparator 118 automatically compares the variants occurred within the same residue and across different residues. The ranking comparator 118 can start comparing the ranking of variant A in Residue 1 with the ranking of variant B in Residue 1 for both models. This ranking comparison can be repeated between variant A in Residue 1 and variant C in Residue 1, variant D in Residue 1, variant A in Residue 2, variant B in Residue 2, variant C in Residue 2, and variant D in Residue 2, respectively. The ranking comparison can also be repeated between variant B in Residue 1 with variant C in Residue 1, variant D in Residue 1, variant A in Residue 2, variant B in Residue 2, variant C in Residue 2, and variant C in Residue 2, respectively. The ranking comparison process can be accompanied by score re-reassignment, where each variant is re-assigned a pathogenicity score generated from the first model 104, based on the ranking of the second model 114. Hence, different variants located at the same microenvironmental context as well as different microenvironmental contexts can be compared, re-ranked and re-assigned with pathogenicity scores.

In another implementation, the score reassignor 128 may maintain the value and scale of each score within the second set of pathogenicity scores 126 but re-assign these scores to the set of variants based on the first score-to-rank mapping 106. For example, variant C within a set of variants has a given pathogenicity score of 0.2 mapped to a corresponding ranking of 2, both generated from the first model 104. The same variant C has a different score of -15.0 and a corresponding ranking of 4, both generated from the second model 114. The ranking comparator 118 compares the two rankings, namely, 2 and 4. The score reassignor 128, based on the ranking of 2 generated from the first model, re-assigns a given score of -20.0 to variant C, where this score was originally assigned to another variant D that had a ranking of 2 generated from the second model 114.

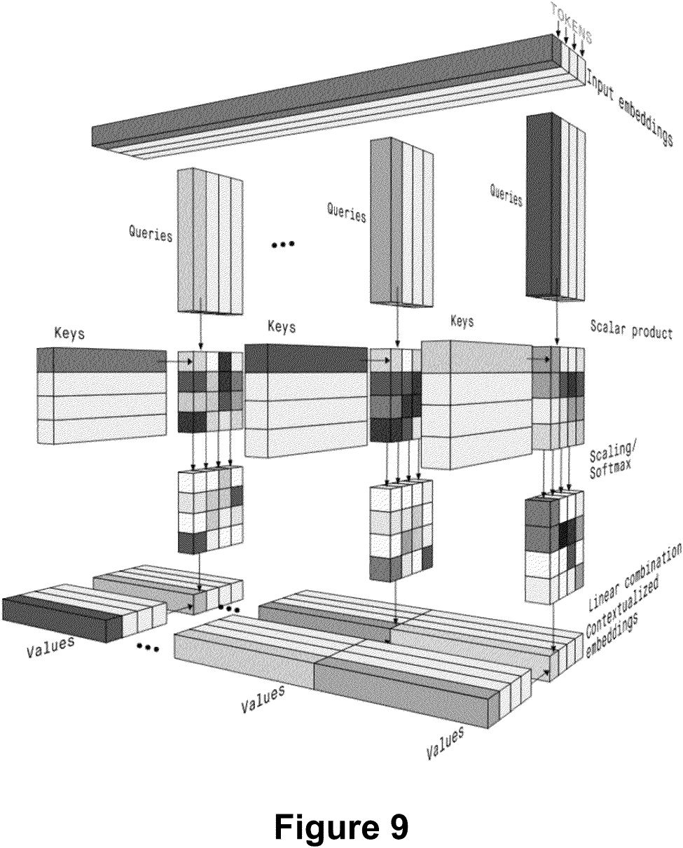

The reassigned pathogenicity scores can also be rescaled. In one implementation, the first set of pathogenicity scores 126 has a first scale, for example, from 0 (benign) to 1 (pathogenic), while the second set of pathogenicity scores 146 has a second scale, for example, from - ∞ (pathogenic) to + ∞ (benign). The system 100 further includes a rescaling logic that rescales pathogenicity scores based on the first and second scales. The rescaling logic will be described in more detail in accordance with FIG. 9.

In another implementation, the score reassignor 128 may re-assign pathogenicity scores to the set of variants based on the first score-to-rank mapping 106 and the second score-to-rank mapping 116. The reassignment logic 108 further includes a combination logic that combines the originally assigned pathogenicity score and the reassigned score, to generate a combined pathogenicity score. The combined pathogenicity score can be an average, a weighted average, or a weighted sum of the originally assigned pathogenicity score and the reassigned score. Consider the aforementioned variants A and B as an example. instead of directly reassigning the score of 0.3 to A, the combination logic combines variant A’s original score of 0.2 and its reassigned score of 0.3 and generates a combined pathogenicity score of 0.25 as an average of the two scores.

Next, we turn to the advantages of the presently disclosed technology by using the pathogenicity scores and rankings generated from one model to re-rank the pathogenicity scores generated from another model and including the re-ranking into the model’s training process. As described above, the two models may use different types of input as their training datasets and therefore generate pathogenicity scores with different scales and values, mapped to different rankings. The two models may also have different architectures with focuses and strengths in different aspects. For example, the first model can be a discriminative model that makes predictions on the unseen data based on conditional probability and can be used either for classification or regression problem statements. The discriminative model can be a site-independent or pairwise interaction model that utilizes evolutionary profiles and 3D structural information of protein sequences primarily from human and primate. Thus, the model excels when predicting the pathogenicity of variants across protein domains and even across proteins, while not effective in determining high-order epistasis. On the other hand, the second model can be a generative model that focuses on the distribution of a dataset to return a probability for a given example. Here, it can be used to quantify the effects of variants in mutations by a ratio of mutant likelihood to a non-mutant (i.e., wildtype) likelihood. The generative model can be a non-linear latent variable model that posts hidden variables to jointly detect global patterns and local patterns of sequence variations across windows spanning multiple positions and multiple sequences in the plurality of aligned sequences. Thus, it may provide epistasis-related information and reliable prediction within a local protein domain, but not in across-domains or across-proteins. In other words, a pathogenicity score in one protein domain may indicate high pathogenicity while the same score in another protein domain may indicate the variant is benign.

The proteome has ten million positions. Accordingly, there are nineteen combinatorically generated amino acid substitutions as variants for each of the ten million positions. Each of the two models can generate a pathogenicity score for each variant located at a particular microenvironmental context. By combining the prediction results from different models, the technology disclosed not only improves in predicting variants across protein domains and across proteins but also in predicting variants in the local mutational neighborhood in the context of high-order epistasis. As an example, the technology disclosed can use the rankings of the variant pathogenicity scores generated from one model and re-rank the scores generated from another model. The rankings generated from one model accurately reflect the effects of the variants in the surrounding context, and the re-ranked scores, generated from another model, remain optimal and consistent across different protein domains and different proteins. The re-ranking processes, as described in more detail below, takes into consideration the context of high-order epistasis and meanwhile, outputs consistent pathogenicity scores. It can effectively avoid undesired situations where the same pathogenicity score indicates high pathogenicity in one protein domain but entirely benign in another.

Before going into further details, we first explain the role of epistasis in the context of protein evolution, and the intuition as to why the prediction of the pathogenicity of variants in the context of both low-order and high-order epistasis is critical.

Mathematically, epistasis is a transform (Ω) in which phenotypes (y̅) of individual variants are represented as context-dependent effects of the underlying mutations (ω ̅):

$$\text{ω} \overline{} = \text{Ω} \overline{\text{y}}$$

For N positions with a single substitution at each position, y̅ is a vector of 2N phenotypic measurements in binary order and ω ̅ is a vector of 2N corresponding epistatic interactions. A first-order epistatic term (ω1) is the phenotypic effect of a single mutation, a second-order epistatic term (ω2) is the degree to which a single mutation effect is different in the background of the second mutation, and a third-order epistasis (ω3) is the degree to which the second-order epistasis is different in the background of a third mutation. Higher-order terms follow the same principle, such that an nth order epistatic term is the degree to which an n-1 order term depends on the context of yet another mutation, comprising a hierarchy of possible couplings between mutations. A key point is that ω ̅ and y̅ contain exactly the same information, but simply differ in its organization; y̅ represents the phenotypes of individual variants while ω ̅ represents the non-additive interactions between the mutations.

Some examples below can help to explain the concept of epistasis. If we take the variant L63M/S 168G/A174L/N207K as an arbitrary reference state (yref = 0.85, blue fluorescence) the data show that introducing the mutation Y197R results in reduced brightness (y = 0.28). The difference in these two values defines a first-order epistasis (ω1 = yY197R-yref = -0.57). However, in the background of F143S, the effect of Y197R is entirely different; it shows increased brightness (ω1|F143S =+ 0.49), with conversion to red fluorescence. This indicates a large second-order epistatic term (ω2 = ω1|F143S—ω1 = 1.06), meaning that the effect of Y197R is context-dependent on F143S. This second-order term is itself dependent on other mutations. For example, in the background of V45A, the second-order epistasis between Y197R and F143S nearly vanishes (ω2|V45A = -0.01), indicating a large third-order epistasis (ω3 = ω2|V45A-ω2 = -1.07). These findings show that Y197R, F143S, and V45A work as a cooperative unit whose contribution to the phenotype cannot be broken down into a simple, additive contribution of the underlying mutational effects. Instead, the prediction of phenotypes involving these mutations requires knowledge of their individual effects and epistatic interactions at all orders.

Understanding the phenotypic effects of variations is a central challenge for bioengineering and basic biology, especially considering the complexity in the sequence-structure-function relationship in a protein space and the context-dependence of the effects of mutations. Contemporary deep learning models have become powerful tools for exploring the evolutionary, structural and functional organization across protein space. Deep learning models have strengths and weaknesses, based on their input for training and validation, as well as model architectures The disclosed technology that combines different models maintains the strengths of different models while avoiding weaknesses of the models when used individually. Therefore, The disclosed technology leads to improvements in predicting the pathogenicity of variants and prediction of phenotypes, both of which require knowledge of individual effects of the variants as well as high-order epistatic interactions.

Next, we turn to the different input that can be used to train, validate and test the two models, and examples of different architectures of the two models.

### First Model

FIG. 2 illustrates an example of the first model using different types of input to a pathogenicity classifier for variant pathogenicity prediction. The first model 200 includes a pathogenicity classifier 204 that uses different types of first input 202 and generates a first pathogenicity score-to-rank mapping 206, including variant pathogenicity scores 216 and variant pathogenicity score ranking 226. As illustrated, the variant pathogenicity score generated from the first model 200 ranges from 0 (benign) to 1 (pathogenic).

In one implementation, when a given protein sequence is used as an input to the first model, for each amino acid located in a given position of the protein sequence, the output is a vector with 20 floating numbers. Each floating number corresponds to the pathogenicity of a particular alternative amino acid as there are 20 types of amino acids.

### Input to First Model

As illustrated in FIG. 2, the first input 202 may include one-hot encodings of a reference target protein sequence 212 and one-hot encodings of an alternative protein sequence with variant amino acid(s) 222. In one implementation, one-hot encodings of the reference target protein sequence 212 represents a known amino acid sequence from human without variants × 20-depth (encoding 20 different amino acids), and one-hot encoding of alternative protein sequences with variant amino acid 222 represents an alternative protein sequence with a variant × 20-depth, where the variant amino acid substitutes an original amino acid (also called a reference amino acid) in the reference target protein sequence. In other words, the reference amino acid and the variant amino acid are located at the same position in the reference target protein sequence and the alternative protein sequence, respectively. Both the reference and the alternative protein sequences are reshaped to a matrix of L×20, where L is the length of the sequence.

The first input 202 may further include three-dimensional (3D) structural information of the reference target protein sequence and the alternative protein sequence 232. Protein sites are microenvironments within a protein structure, distinguished by their structural or functional role. A site can be defined by a 3D location and a local neighborhood around this location in which the structure or function exists. Since it has been established that structure is far more conserved than sequence, the protein structural data provides an opportunity to systematically study the underlying pattern governing the structural-functional relationships using data-driven approaches.

In one implementation, proteins are represented by a collection of atoms and their coordinates in 3D space. An amino acid has a variety of atoms, such as carbon atoms, oxygen (O) atoms, nitrogen (N) atoms, and hydrogen (H) atoms. The atoms can be further classified as side-chain atoms and backbone atoms. The backbone carbon atoms can include alpha-carbon (Cα) atoms and beta-carbon (Cβ) atoms. The 3D atomic coordinates can be attributed to 21 amino acid categories, including 20 categories of amino acid with a similar basic structure but differ in the side chains and 1 stop or gap amino acid category. For example, an amino acid-wise classification of alpha-carbon atoms can respectively list alpha-carbon atoms or beta-carbon atoms under each of the 21 amino acid categories. In other examples, an amino acid-wise classification of oxygen atoms can respectively list oxygen atoms, nitrogen atoms or hydrogen atoms under each of the 21 amino acid categories.

In another implementation, the 3D structural information of the reference target protein sequence and the alternative protein sequence 232 includes voxelized features. For example, a voxel grid is centered at the reference amino acid experiencing a target variant at the amino acid level. In particular, the voxel grid can be centered at an atomic coordinate of a particular atom of the reference amino acid experiencing the target variant. The voxel grid can be centered at the 3D atomic coordinate of one of the alpha-carbon atoms, beta-carbon atoms, oxygen atoms, nitrogen atoms, or hydrogen atoms of the reference amino acid experiencing the target variant. The voxel grid can have any resolution, for example, 3×3×3, 5×5×5, 7×7×7, and so on. Voxels in the voxel grid can be of any size, for example, one angstrom (Å) on each side, two Å on each side, three Å on each side, and so on.

The voxels in the voxel grid can have a plurality of channels (or features). In one implementation, the voxels in the voxel grid have a plurality of distance channels, for example, twenty-one distance channels for the twenty-one amino acid categories, respectively (including a stop or gap amino acid category). The distance channels are independently generated for each of the twenty-one amino acid categories. Consider amino acid Alanine (A) category using a 3×3×3 voxel grid with 27 voxels as an example. An Alanine distance channel includes 27 distance values for the 27 voxels in the voxel grid, respectively. The 27 distance values are measured from respective centers of the 27 voxels in the voxel grid to the respective nearest atoms (e.g., alpha-carbon (Cα) atoms, beta-carbon (Cβ) atoms, oxygen (O) atoms, nitrogen (N) atoms, and hydrogen (H) atoms) in the Alanine amino acid category. The distance values are measured between the 3D coordinates of the voxel centers and the 3D atomic coordinates of the atoms.

Yet in another implementation, the 3D structural information of the reference target protein sequences and the alternative protein sequences 232 includes a “gapped spatial representation” of a protein that excludes at least one gap amino acid in the protein. “Spatial representation” of a protein characterizes structural information about amino acids in the protein, based on the shapes, locations, positions, patterns, and/or arrangement of the amino acids. Amino acid-wise distance channels as discussed above can be used as a spatial representation of a protein. When a particular amino acid in the protein is removed or masked from the protein, this amino acid is called a “gap amino acid”, and the resulting protein that lacks the gap amino acid is called a “gapped protein” or a “vacancy-containing protein.” Therefore, “gapped spatial representation” is such a spatial representation of a vacancy-containing protein. In one implementation, when generating a gapped spatial representation, a gap amino acid is “masked” by excluding (or not considering or ignoring) one or more atoms or atom-types of the gap amino acid. The spatial representations of non-gap amino acids are encoded as amino acid class-wise distance channels, where each of the amino acid class-wise distance channels has voxel-wise distance values for voxels. The voxel-wise distance values specify distances from the corresponding voxels in a plurality of voxels to the atoms of non-gap amino acids. The spatial representations of non-gap amino acids are determined based on spatial proximity between the corresponding voxels and the atoms of these non-gap amino acids. However, the spatial representations of the gap amino acid are excluded from the gapped spatial representation by disregarding distances from the corresponding voxels to atoms of the gap amino acid when determining the voxel-wise distance values.

In one implementation, when a reference gap amino acid at a given position in a reference target protein sequence is excluded, a target alternate amino acid may fill the vacancy at the given position. The pathogenicity classifier 204 can use both the spatial representations of the vacancy-containing protein (i.e., the “gapped spatial representation”) and the target alternate amino acid as input to predict a pathogenicity score for the target alternate amino acid. In another implementation, each of the 20 amino acid categories can be used as a target alternate amino acid that fills the vacancy at the given position in the reference target protein sequence. Thus, the pathogenicity classifier 204 can predict a pathogenicity score for each of the 20 amino acid categories (e.g., by using a 20-way softmax classification).

As illustrated in FIG. 2, the first input 202 may further include evolutionary profiles of a target protein sequence 242. For example, the evolutionary profiles of the target protein sequence include evolutionary conservation summary statistics of amino acids in the target protein sequence 252. Evolutionary conservation refers to the presence of similar genes, portions of genes, or chromosome segments in different species, reflecting both the common origin of species and important functional property of the conserved elements. Mutations occur spontaneously in each generation, randomly changing an amino acid here and there in a protein. Individuals with mutations that impair critical functions of proteins may have resulting problems that make them less able to reproduce. Harmful mutations are lost from the gene pool because the individuals carrying them reproduce less effectively. Since the harmful mutations are lost, the amino acids critical for the function of a protein are conserved in the gene pool. In contrast, harmless (benign or very rarely, beneficial) mutations are kept in the gene pool, producing variability in non-critical amino acids. Evolutionary conservation in proteins is identified by aligning the amino acid sequences of proteins with the same function from different taxa (orthologs). Predicting the functional consequences of variants relies at least in part on the assumption that crucial amino acids for protein families are conserved through evolution due to negative selection (i.e., amino acid changes at these sites were deleterious in the past), and that mutations at these sites have an increased likelihood of being pathogenic (causing disease) in human. In general, homologous sequences of a target protein are collected and aligned, and a metric of conservation is computed based on the weighted frequencies of different amino acids observed in the target position in the alignment.

In one implementation, the evolutionary profiles of a target protein sequence include position weight matrix (PWM) conservation profiles calculated from a plurality of species. For example, to predict a pathogenicity score of a target variant in a protein sequence from human, three different position weight matrices (PWMs) can be generated from multiple sequence alignments (MSA) of 99 vertebrates for this target variant, including one for 12 primates, one for 47 mammals excluding primates, and one for 40 vertebrates excluding primates and mammals. Each of the PWMs has a dimension of L × 20, where L is the length of flanking sequences around the target variant. It comprises counts of amino acids seen in each category of species.

In another implementation, the evolutionary profiles of the target protein sequence include position-specific frequency matrices (PSFMs) 262 and position-specific score matrices (PSSMs) 272. PSFMs represent the conservation of amino acids in a human protein sequence across aligned protein sequences of other species by determining, on a location-by-location basis, the frequency of occurrence of an amino acid in the human protein sequence across the aligned protein sequences of other species. On the other hand, PSSMs represent the evolutionary conservation of alternate amino acids that are candidates for filling an amino acid vacancy in a vacancy-containing protein. PSSMs represent evolutionary conservation of alternate amino acids based at least in part on structural (or spatial) compatibility between substitute amino acids and adjacent amino acids in a neighborhood of the amino acid vacancy (e.g., the right and left flanking amino acids around the amino acid vacancy). PSFMs and PSSMs can be used to identify the evolutionary conservation profiles of homologous proteins of the plurality of species and differentiate most-conserved amino acids from non-conserved amino acids.

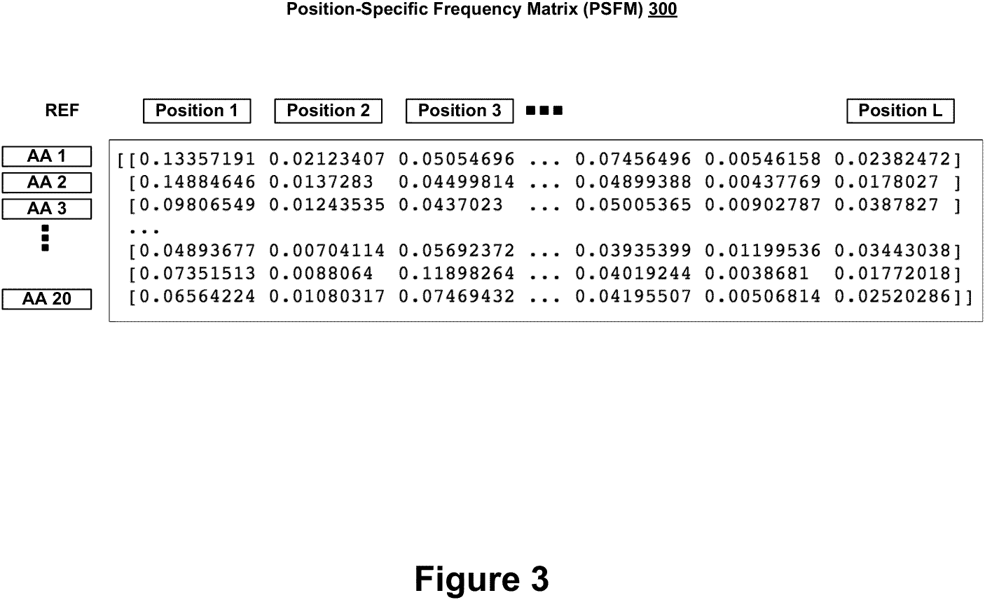

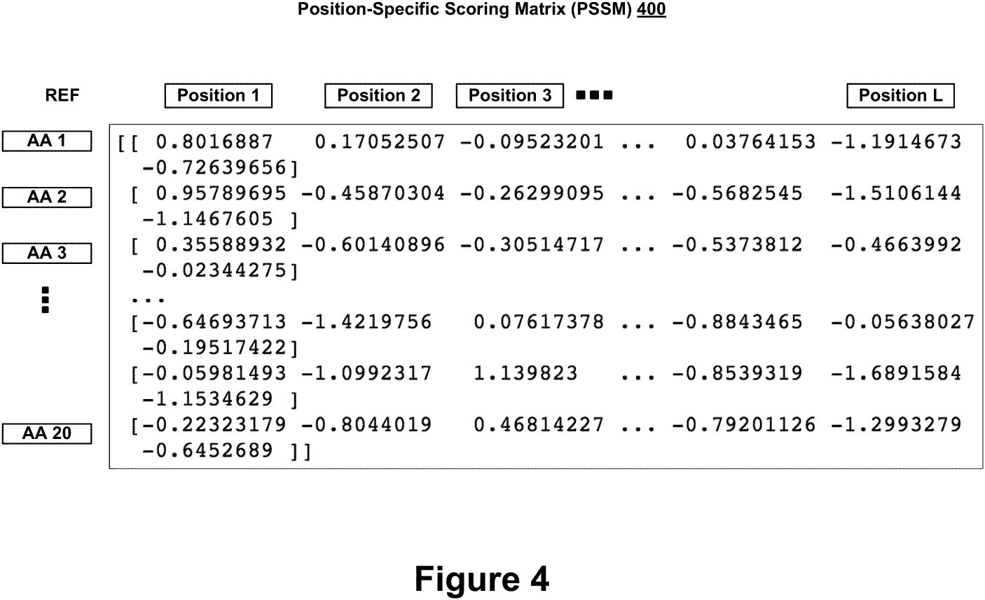

FIG. 3 illustrates an example position-specific frequency matrix (PSFM) 300. FIG. 4 illustrates an example input position-specific scoring matrix (PSSM) 400. Sequence profiles of a protein sequence X of a length L are an L × 20 matrix, either in the form of a PSFM or a PSSM. The columns of a PSFM and a PSSM are indexed by the alphabet of amino acids (AA1, AA2, ..., AA20 as illustrated) and each row corresponds to a position in the protein sequence (Position 1, Position 2, ..., Position L as illustrated). PSFMs and PSSMs contain the frequencies and the substitution scores, respectively, of the amino acids at different positions in the protein sequence. As illustrated in FIG. 3, each row of a PSFM is normalized to sum to 1. The sequence profiles of the protein sequence X are computed by aligning X with multiple sequences in a protein database that has statistically significant sequence similarities with X. Therefore, the sequence profiles contain more general evolutionary and structural information of the protein family that protein sequence X belongs to, and thus, provide valuable information for remote homology detection and fold recognition.

A protein sequence (e.g., a reference protein sequence) can be used as a seed to search and align homogenous sequences from a protein database (e.g., SWISSPROT) using, for example, a PSI-BLAST program. The aligned sequences share some homogenous segments and belong to the same protein family. The aligned sequences are further converted into two profiles, namely, PSSM and PSFM, to express their homogeneous information. Consider in FIGS. 3 and 4, the column of amino acids (AA1, AA2, ..., AA20) is referred to as “i” and the position in the protein sequence (Position 1, Position 2, ..., Position L) is referred to as “j”. A PSFM contains the weighted observation frequencies of each position of the aligned sequences. Specifically, the (i, j)-th entry of the PSFM matrix represents the possibility of having amino acid type i in the j-th position of the query sequence. Within a PSSM, each column represents a log-likelihood of the residue substitutions at the corresponding positions in the query sequence. Specifically, the (i, j)-th entry of the PSSM matrix represents the chance of the amino acid in the j-th position of the query sequence being mutated to amino acid type i during the evolution process.

The first input 202 may further include the secondary structure and solvent accessibility of the reference target protein sequence and the alternative protein sequence. For example, a three-state secondary structure profile at each amino acid position of the protein sequence can be represented as alpha helix (H), beta sheet (B), and coils (C). A three-state solvent accessibility profile at each amino acid position of the protein sequence can be represented as buried (B), intermediate (I), and exposed (E). In one implementation, the secondary structure and solvent accessibility of the reference target protein sequence and alternative protein sequence are predicted by other deep learning networks, and the predicted results are used here as indirect input to the pathogenicity classifier 204. Given the small number of human proteins that have been successfully crystallized, inferring structure from the primary sequence and using it as an input to the pathogenicity classifier 204 has the advantage of avoiding biases due to incomplete protein structure and functional domain annotations.

### The Architecture of First Model

In one implementation, the first model 200 is a deep neural network, for example, convolutional neural network (CNN) or recurrent neural network (RNN), that uses multiple non-linear and complex transforming layers to successively model high-level features. Given that sequenced data (e.g., DNA or RNA sequence, amino acid sequence) can be multi- and high-dimensional, deep neural networks have great promise for bioinformatics research because of their broad applicability and enhanced prediction power. In particular, convolutional neural networks have been adapted to solve sequence-based problems in genomics such as motif discovery, pathogenic variant identification, and gene expression inference. CNNs use a weight-sharing strategy that is especially useful for studying DNA because it can capture sequence motifs, which are short, recurring local patterns in DNA that are presumed to have significant biological functions.

In one implementation, the first model 200 is a deep neural network that predicts the pathogenicity of missense variants from protein sequence and sequence conservation data (See Sundaram, L. et al. Predicting the clinical impact of human mutation with deep neural networks. Nat. Genet. 50, 1161-1170 (2018), referred to herein as “PrimateAI”). PrimateAI uses deep neural networks trained on variants of known pathogenicity with data augmentation using cross-species information. In particular, PrimateAI uses sequences of wild-type and mutant proteins to compare the difference and decide the pathogenicity of mutations using the trained deep neural networks. PrimateAI learns important protein domains, conserved amino acid positions, and sequence dependencies directly from the training data consisting of about 120,000 human samples.

In one implementation, the inputs to PrimateAI include one-hot-encoding of a reference target protein sequence, one-hot-encoding of an alternative protein sequence with a target variant, evolutionary profiles represented by three PWMs from primate, mammal, and vertebrate alignments, respectively, as well as the prediction of the secondary structure and solvent accessibility of the reference target protein sequence. The pathogenicity classifier 204 of PrimateAI is a convolutional neural network that uses different types of convolutions such as 1D convolutions, 2D convolutions, 3D convolutions, dilated or atrous convolutions, transposed convolutions, separable convolutions, and depthwise separable convolutions. Some layers also use ReLU activation function which greatly accelerates the convergence of stochastic gradient descent compared to saturating non-linearities such as sigmoid or hyperbolic tangent. Other examples of activation functions that can be used in PrimateAI include parametric ReLU, leaky ReLU, GeLU, and exponential linear unit (ELU).

Some layers in PrimateAI also use batch normalization. Regarding batch normalization, the distribution of each layer in a CNN changes during training and it varies from one layer to another. This reduces the convergence speed of the optimization algorithm. Batch normalization is a technique to overcome this problem. Denoting the input of a batch normalization layer with x and its output using z, batch normalization applies the following transformation on x:

$$z = \frac{x - μ}{\sqrt{{σ}^{2} + ∈}} γ + β$$

Batch normalization applies mean-variance normalization on the input x using µ and σ and linearly scales and shifts it using γ and β. The normalization parameters µ and σ are computed for the current layer over the training set using a method called exponential moving 15 average. In other words, they are not trainable parameters. In contrast, γ and β are trainable parameters. The values for µ and σ calculated during training are used in forward pass during inference.

In another implementation, the first model 200 is a deep neural network to predict variant pathogenicity based on 3D structural information of protein sequences, referred to herein as “PrimateAI 3D”. In one implementation, PrimateAI 3D uses as input voxelized one-hot-encodings of amino acids in a reference target protein sequence and one-hot-encodings of amino acids in an alternative protein sequence with a target variant. PrimateAI 3D also uses as input twenty-one amino acid-wise distance channels, where each amino acid-wise distance channel includes a distance value for each of the 27 voxels within a 3×3×3 voxel grid. In another implementation, PrimateAI 3D also uses as input the evolutionary profiles of the reference target protein sequence, for example, PWMs and PSFMs as described above.

In one implementation, PrimateAI 3D is a cascade of processing modules, each of which can include 1D convolutions, 2D convolutions, 3D convolutions, 4D convolutions, 5D convolutions, dilated or atrous convolutions, transpose convolutions, depthwise separable convolutions, pointwise convolutions, 1 × 1 convolutions, group convolutions, flattened convolutions, spatial and cross-channel convolutions, shuffled grouped convolutions, spatial separable convolutions, and deconvolutions. It can use one or more loss functions such as logistic regression/log loss, multi-class cross-entropy/softmax loss, binary cross-entropy loss, mean-squared error loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). It can include upsampling layers, downsampling layers, recurrent connections, gates, and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, attention mechanisms, and gaussian error linear unit. The processing modules may also include a final softmax layer that produces exponentially normalized scores for the target variant belonging to a benign class and a pathogenic class.

In yet another implementation, the first model 200 is a deep neural network to predict variant pathogenicity based on “gapped spatial representation” of a vacancy-containing protein that excludes at least one gap amino acid in the protein, and a spatial representation of an alternate amino acid. The first model 200 is referred to herein as “JigsawAI”. The spatial representation of the vacancy-containing protein can be realized by using voxelized amino acid category-wise distance calculations that exclude the reference gap amino acid (or atoms thereof). In one implementation, an alternate amino acid may fill the vacancy at the given position in the reference target protein sequence. The representation of the alternate amino acid, along with the “gapped spatial representation” of the vacancy-containing protein are input to the pathogenicity classifier which generates an output pathogenicity score of the alternate amino acid. In another implementation, JigsawAI also uses evolutionary profiles of protein sequences as input.

In one implementation, the pathogenicity classifier 204 of JigsawAI is a cascade of processing modules, each of which can include 1D convolutions (1 × 1 × 1 CONV), 3D convolutions (3×3×3 CONV), ReLU non-linearity, and batch normalization (BN). Other examples of the processing modules include fully-connected layers, a dropout layer, a flattening layer, and a final softmax layer that produces exponentially normalized scores for the target variant belonging to a benign class and a pathogenic class.

A person skilled in the art will appreciate that for the architecture of the first model, other current and future artificial intelligence, machine learning, and deep learning models, datasets, and training techniques can be incorporated into the disclosed model without deviating from the spirit of the technology disclosed.

### Second Model

As described above, the second model can have a different architecture from the first network. For example, the second model can be a generative model that uses probability estimates and likelihood to model data points and differentiate between different class labels present in a dataset. Generative models are often used in natural language processing. In recent years, generative models have emerged as powerful machine-learning tools to discover evolutionary, structural and functional information across protein space and thus, referred to as protein language models. The second model can be a protein language model that is trained on large and diverse protein sequence databases, and captures information of the sequence-structure-function relationship of the protein including, e.g., the pathogenicity of variants in the context of epistasis.

In one implementation, the second model can capture higher-order epistatic interactions between mutations that span multiple positions and multiple sequences in the MSA. In another implementation, the higher-order epistatic interactions between the mutations may form an undirected graph trajectory. The second model can use an undirected graphical model to identify highly evolutionarily coupled loci given the MSA. See, Genome-wide discovery of epistatic loci affecting antibiotic resistance using evolutionary couplings, Benjamin Schubert, Rohan Maddamsetti, Jackson Nyman, Debora S. Marks, Nature Microbiology, 4, pages328-338 (2019). In another implementation, the second model can capture two-way epistatic interactions (e.g., pairwise level) between mutations. In yet another implementation, the second model can capture higher-order (e.g., three-way, four-way) epistatic interactions that describe the full extent of possible interactions.

In one implementation, the second model processes the MSA as an input and generates a reconstruction of the MSA as an output. For a given alternate protein sequence, the second model may generate a reconstruction of the alternate protein sequence as a base-wise probability score for each amino acid in the protein sequence. In other implementations, a joint probability is determined from the base-wise probability score corresponding to each amino acid and, in turn, is used as a pathogenicity score for a given variant that mutates the reference target protein sequence to the given alternate protein sequence.

In yet another implementation, the second model is pre-trained. In other words, the respective coefficients and latent space configurations of the second model are pre-trained to process and reconstruct the MSA that has a respective reference target protein sequence as a query sequence. For the given reference target protein sequence, the second model may have a particular or preset coefficient and latent space configuration. Considering there are twenty thousand reference protein sequences in human proteome, the second model may accordingly have a particular or preset coefficient and latent space configuration for each of the twenty thousand reference protein sequences. When a given protein sequence is used as an input to the second model, for each amino acid located in a given position of the protein sequence, the output is a vector with 20 floating numbers. Each floating number corresponds to the pathogenicity of a particular alternative amino acid as there are 20 types of amino acids.

In one implementation, the second model is a multilayer perceptron (MLP). In another implementation, the second model is a feedforward neural network. In yet another implementation, the second model is a fully-connected neural network. In a further implementation, the second model is a fully convolution neural network. In yet further implementation, the second model is a semantic segmentation neural network. In yet another further implementation, the second model is a generative adversarial network (GAN) (e.g., CycleGAN, StyleGAN, pixelRNN, text-2-image, DiscoGAN, IsGAN).

In one implementation, the second model is a convolution neural network (CNN) with a plurality of convolution layers. In another implementation, the second model is a recurrent neural network (RNN) such as a long short-term memory network (LSTM), bi-directional LSTM (Bi-LSTM), or a gated recurrent unit (GRU). In yet another implementation, the second model includes both a CNN and an RNN.

In yet other implementations, the second model can use 1D convolutions, 2D convolutions, 3D convolutions, 4D convolutions, 5D convolutions, dilated or atrous convolutions, transpose convolutions, depthwise separable convolutions, pointwise convolutions, 1 × 1 convolutions, group convolutions, flattened convolutions, spatial and cross-channel convolutions, shuffled grouped convolutions, spatial separable convolutions, and deconvolutions. The second model can use one or more loss functions such as logistic regression/log loss, multi-class cross-entropy/softmax loss, binary cross-entropy loss, mean-squared error loss, L1 loss, L2 loss, smooth L1 loss, and Huber loss. The second model can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The second model can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential linear unit (ELU), sigmoid and hyperbolic tangent (tanh)), batch normalization layers, regularization layers, dropout, pooling layers (e.g., max or average pooling), global average pooling layers, and attention mechanisms (e.g., self-attention).

The second model can be a rule-based model, linear regression model, a logistic regression model, an Elastic Net model, a support vector machine (SVM), a random forest (RF), a decision tree, and a boosted decision tree (e.g., XGBoost), or some other tree-based logic (e.g., metric trees, kd-trees, R-trees, universal B-trees, X-trees, ball trees, locality sensitive hashes, and inverted indexes). The second model can be an ensemble of multiple models, in some implementations.

The second model is trained using backpropagation-based gradient update techniques. Example gradient descent techniques that can be used for training the second model include stochastic gradient descent, batch gradient descent, and mini-batch gradient descent. Some examples of gradient descent optimization algorithms that can be used to train the second model is Momentum, Nesterov accelerated gradient, Adagrad, Adadelta, RMSprop, Adam, AdaMax, Nadam, and AMSGrad.

### Input to Second Model

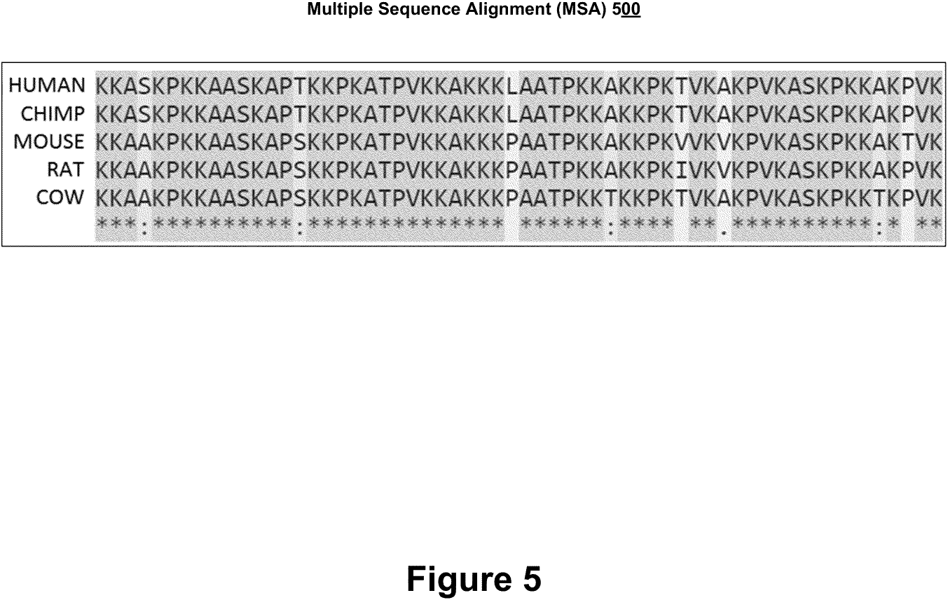

In one implementation, input to the second model includes epistasis-related information expressed by amino acid patterns spanning a target protein sequence and a plurality of non-target protein sequences aligned with the target protein sequence in MSA. The amino acid patterns may be variant or mutation patterns and may include multiple, e.g., at least three amino acids. In another implementation, input to the second model includes sequence information of a target protein sequence and a plurality of non-target protein sequences that are homologous with the target protein sequence. FIG. 5 illustrates an example of MSA 500. It is noted that FIG. 5 only lists one target protein sequence from human aligned with four non-target protein sequences from chimpanzee, mouse, rat, and cow, respectively. A person skilled in the art will appreciate MSA 500 could include more non-target homologous protein sequences, for example, at least one hundred, five hundred, one thousand sequences.

In one implementation, the target protein sequence belongs to a target species (e.g., human), and the non-target protein sequences belong to a group of species that shares a genus with the target species. The shared genus is, for example, Genus Homo. In another implementation, the non-target protein sequences belong to a group of species that shares a family with the target species. The shared family is, for example, hominids. In yet another implementation, the non-target protein sequences belong to a group of species that shares a class with the target species. The shared family is, for example, mammals. In yet another implementation, the non-target protein sequences belong to a group of species that shares a phylum with the target species. The shared phylum is, for example, chordates. In yet another implementation, the non-target protein sequences belong to a group of species that shares a kingdom with the target species. The shared kingdom is, for example, animals.

### The Architecture of Second Model

The following discussion provides different examples of machine learning architectures that can be used to implement the second model. The following example machine learning architectures can take as input machine-processable or vectorized representations of sequence data, for example, one-hot encodings of nucleotides and/or amino acids, process the machine-processable representations through a plurality of hidden layers and weights of the machine learning architectures, produce learned or alternative or intermediate or compressed representations of the machine-processable representations, and generate one or more outputs based on the learned or alternative or intermediate or compressed representations. These outputs can be genotype predictions identifying one or more attributes or identifies of the genomic data, such as the identity of the nucleotides and/or amino acids, evolutionary conservation states of the nucleotides and/or amino acids, the pathogenicity of the nucleotides and/or amino acids, and so on.

We start from example architectures of generative models. Generative models are often used in natural language processing. In recent years, generative models have emerged as powerful machine-learning tools to discover evolutionary, structural and functional information across protein space and thus, referred to as protein language models. The second model, as a protein language model, can be trained on large and diverse protein sequence databases, and captures information of the sequence-structure-function relationship of the protein including, e.g., the pathogenicity of variants in the context of epistasis.

### Transformer-Based Models

In one implementation, the second model is a Transformer model that relies on a self-attention mechanism to compute a series of context-informed vector-space representations of elements in the input sequence and the output sequence, which are then used to predict distributions over subsequent elements as the model predicts the output sequence element-by-element. Not only is this mechanism straightforward to parallelize, but as each input’s representation is also directly informed by all other inputs’ representations, this results in an effectively global receptive field across the whole input sequence. This stands in contrast to, e.g., convolutional architectures which typically only have a limited receptive field.

In different implementations, the second model can include self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, BERT, SpanBERT, RoBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCiT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN + FPN, DETR-DC5, TSP-FCOS, TSP-RCNN, ACT+MKDD (L=32), ACT+MKDD (L=16), SMCA, Efficient DETR, UP-DETR, UP-DETR, ViTB/16-FRCNN, ViT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

### Transformer Logic

Machine learning is the use and development of computer systems that can learn and adapt without following explicit instructions, by using algorithms and statistical models to analyze and draw inferences from patterns in data. Some of the state-of-the-art models use Transformers, a more powerful and faster model than neural networks alone. Neural networks process input in series (e.g., time series data including sequencing-by-synthesis (SBS) sequencing data) and weight relationships by distance in the series. Transformers can process input in parallel and do not necessarily weight by distance. Transformers can be used in addition to neural networks. This architecture is described here.

### Encoder-Decoder Architecture

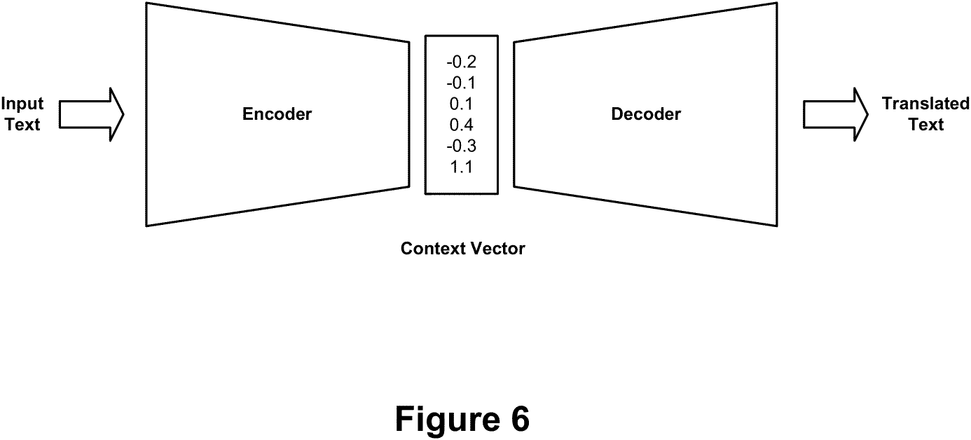

FIG. 6 is a schematic representation of an encoder-decoder architecture. This architecture is often used for time-series data processing (e.g., sequencing data generated via sequencing-by-synthesis) and has two main building blocks. The first building block is the encoder that encodes an input (e.g., nucleotide sequences and/or amino acid sequences) into a fixed-size vector. In the system we describe here, the encoder is based on a recurrent neural network (RNN). At each time step, t, a hidden state of time step, t-1, is combined with the input value at time step t to compute the hidden state at timestep t. The hidden state at the last time step, encoded in a context vector, contains relationships encoded at all previous time steps.

The context vector is then passed to the second building block, the decoder. Conditioned on the input context vector, the decoder generates an output sequence. At each time step, t, the decoder is fed the hidden state of time step, t-1, and the output generated at time step, t-1. The first hidden state in the decoder is the context vector, generated by the encoder.

The whole model is optimized end-to-end by using backpropagation, a method of training a neural network in which the initial system output is compared to the desired output and the system is adjusted until the difference is minimized. In backpropagation, the encoder is trained to extract the right information from the input sequence, the decoder is trained to capture the grammar and vocabulary of the output language. This results in a fluent model that uses context and generalizes well. When training an encoder-decoder model, the real output sequence is used to train the model to prevent mistakes from stacking. When testing the model, the previously predicted output value is used to predict the next one.

When performing a translation task using the encoder-decoder architecture, all information about the input sequence is forced into one vector, the context vector. Information connecting the beginning of the sentence with the end is lost, the vanishing gradient problem. Also, different parts of the input sequence are important for different parts of the output sequence, information that cannot be learned using only RNNs in an encoder-decoder architecture.

### Attention Mechanism

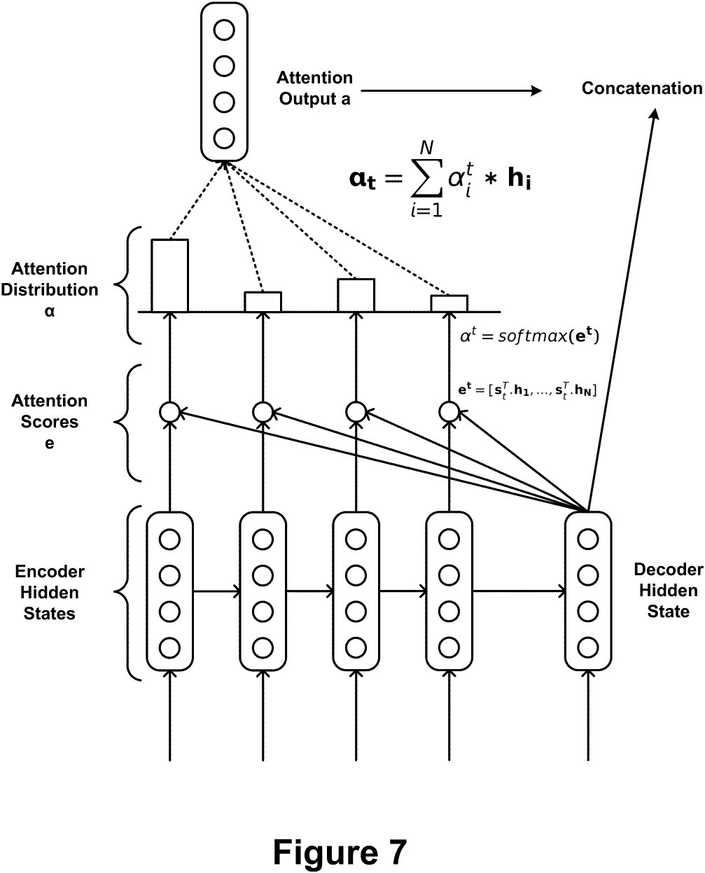

Attention mechanisms distinguish Transformers from other machine learning models. The attention mechanism provides a solution for the vanishing gradient problem. FIG. 7 shows an overview of an attention mechanism added onto an RNN encoder-decoder architecture. At every step, the decoder is given an attention score, e, for each encoder hidden state. In other words, the decoder is given weights for each relationship between words in a sentence. The decoder uses the attention score concatenated with the context vector during decoding. The output of the decoder at time step t is be based on all encoder hidden states and the attention outputs. The attention output captures the relevant context for time step t from the original sentence. Thus, words at the end of a sentence may now have a strong relationship with words at the beginning of the sentence. In the sentence “The quick brown fox, upon arriving at the doghouse, jumped over the lazy dog,” fox and dog can be closely related despite being far apart in this complex sentence.

To weight encoder hidden states, a dot product between the decoder hidden state of the current time step, and all encoder hidden states, is calculated. This results in an attention score for every encoder hidden state. The attention scores are higher for those encoder hidden states that are similar to the decoder hidden state of the current time step. Higher values for the dot product indicate the vectors are pointing more closely in the same direction. The attention scores are converted to fractions that sum to one using the SoftMax function.

The SoftMax scores provide an attention distribution. The x-axis of the distribution is position in a sentence. The y-axis is attention weight. The scores show which encoder hidden states are most closely related. The SoftMax scores specify which encoder hidden states are the most relevant for the decoder hidden state of the current time step.

The elements of the attention distribution are used as weights to calculate a weighted sum over the different encoder hidden states. The outcome of the weighted sum is called the attention output. The attention output is used to predict the output, often in combination (concatenation) with the decoder hidden states. Thus, both information about the inputs, as well as the already generated outputs, can be used to predict the next outputs.

By making it possible to focus on specific parts of the input in every decoder step, the attention mechanism solves the vanishing gradient problem. By using attention, information flows more directly to the decoder. It does not pass through many hidden states. Interpreting the attention step can give insights into the data. Attention can be thought of as a soft alignment. The words in the input sequence with a high attention score align with the current target word. Attention describes long-range dependencies better than RNN alone. This enables analysis of longer, more complex sentence.

The attention mechanism can be generalized as: given a set of vector values and a vector query, attention is a technique to compute a weighted sum of the vector values, dependent on the vector query. The vector values are the encoder hidden states, and the vector query is the decoder hidden state at the current time step.

The weighted sum can be considered a selective summary of the information present in the vector values. The vector query determines on which of the vector values to focus. Thus, a fixed-size representation of the vector values can be created, in dependence upon the vector query.

The attention scores can be calculated by the dot product, or by weighting the different values (multiplicative attention).

### Embeddings

For most machine learning models, the input to the model needs to be numerical. The input to a translation model is a sentence, and words are not numerical. Multiple methods exist for the conversion of words into numerical vectors. These numerical vectors are called the embeddings of the words. Embeddings can be used to convert any type of symbolic representation into a numerical one.

Embeddings can be created by using one-hot encoding. The one-hot vector representing the symbols has the same length as the total number of possible different symbols. Each position in the one-hot vector corresponds to a specific symbol. For example, when converting colors to a numerical vector, the length of the one-hot vector would be the total number of different colors present in the dataset. For each input, the location corresponding to the color of that value is one, whereas all the other locations are valued at zero. This works well for working with images. For natural language processing (NLP), this becomes problematic, because the number of words in a language is very large. This results in enormous models and the need for a lot of computational power. Furthermore, no specific information is captured with one-hot encoding. From the numerical representation, it is not clear that orange and red are more similar than orange and green. For this reason, other methods exist.

A second way of creating embeddings is by creating feature vectors. Every symbol has its specific vector representation, based on features. With colors, a vector of three elements could be used, where the elements represent the amount of yellow, red, and/or blue needed to create the color. Thus, all colors can be represented by only using a vector of three elements. Also, similar colors, have similar representation vectors.

Embedding based on context can be trained. Words with similar meanings occur in similar contexts. At nucleotide level, particular combinations of three DNA or RNA nucleotides correspond to specific amino acids or stop signals during protein synthesis. In addition, homologous proteins or genes have sequence similarity that reflects common ancestry. Different methods take the context into account. For natural language process, some methods, like GloVe, base their context embedding on co-occurrence statistics from corpora (large texts) such as Wikipedia. Words with similar co-occurrence statistics have similar word embeddings. Other methods use neural networks to train the embeddings. For example, they train their embeddings to predict the word based on the context (Common Bag of Words), and/or to predict the context based on the word (Skip-Gram). Training these contextual embeddings is time intensive. For this reason, pre-trained libraries exist. Other deep learning methods can be used to create embeddings. For example, the latent space of a variational autoencoder (VAE) can be used as the embedding of the input. Another method is to use 1D convolutions to create embeddings. This causes a sparse, high-dimensional input space to be converted to a denser, low-dimensional feature space.

### Self Attention: Queries (Q), Keys (K), Values (V)

Transformer models are based on the principle of self-attention. Self-attention allows each element of the input sequence to look at all other elements in the input sequence and search for clues that can help it to create a more meaningful encoding. It is a way to look at which other sequence elements are relevant for the current element. The Transformer can grab context from both before and after the currently processed element.

When performing self-attention, three vectors need to be created for each element of the encoder input: the query vector (Q), the key vector (K), and the value vector (V). These vectors are created by performing matrix multiplications between the input embedding vector using three unique weight matrices.

After this, self-attention scores are calculated. When calculating self-attention scores for a given element, the dot products between the query vector of this element and the key vectors of all other input elements are calculated. To make the model mathematically more stable, these self-attention scores are divided by the root of the size of the vectors. This has the effect of reducing the importance of the scalar thus emphasizing the importance of the direction of the vector. Just as before, these scores are normalized with a SoftMax layer. This attention distribution is then used to calculate a weighted sum of the value vectors, resulting in a vector z for every input element. In the attention principle explained above, the vector to calculate attention scores and to perform the weighted sum was the same, in self-attention two different vectors are created and used. As the self-attention needs to be calculated for all elements (thus a query for every element), one formula can be created to calculate a Z matrix. The rows of this Z matrix are the z vectors for every sequence input element, giving the matrix a size length sequence dimension QKV.

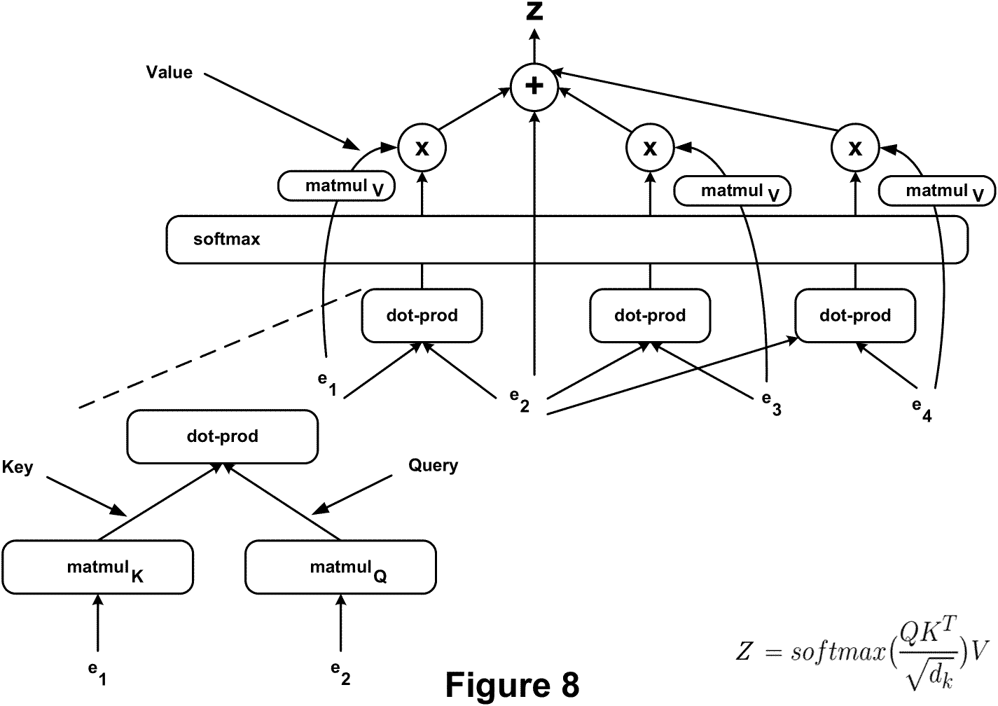

Multi-headed attention is executed in the Transformer. FIG. 8 is a schematic representation of the calculation of self-attention showing one attention head. For every attention head, different weight matrices are trained to calculate Q, K, and V. Every attention head outputs a matrix Z. Different attention heads can capture different types of information. The different Z matrices of the different attention heads are concatenated. This matrix can become large when multiple attention heads are used. To reduce dimensionality, an extra weight matrix W is trained to condense the different attention heads into a matrix with the same size as one Z matrix. This way, the amount of data given to the next step does not enlarge every time self-attention is performed.

When performing self-attention, information about the order of the different elements within the sequence is lost. To address this problem, positional encodings are added to the embedding vectors. Every position has its unique positional encoding vector. These vectors follow a specific pattern, which the Transformer model can learn to recognize. This way, the model can consider distances between the different elements.

As discussed above, in the core of self-attention are three objects: queries (Q), keys (K), and values (V). Each of these objects has an inner semantic meaning of their purpose. One can think of these as analogous to databases. We have a user-defined query of what the user wants to know. Then we have the relations in the database, i.e., the values which are the weights. More advanced database management systems create some apt representation of its relations to retrieve values more efficiently from the relations. This can be achieved by using indexes, which represent information about what is stored in the database. In the context of attention, indexes can be thought of as keys. So instead of running the query against values directly, the query is first executed on the indexes to retrieve where the relevant values or weights are stored. Lastly, these weights are run against the original values to retrieve data that are most relevant to the initial query.

FIG. 9 depicts several attention heads in a Transformer block. We can see that the outputs of queries and keys dot products in different attention heads are differently colored. This depicts the capability of the multi-head attention to focus on different aspects of the input and aggregate the obtained information by multiplying the input with different attention weights.

Examples of attention calculation include scaled dot-product attention and additive attention. There are several reasons why scaled dot-product attention is used in the Transformers. Firstly, the scaled dot-product attention is relatively fast to compute, since its main parts are matrix operations that can be run on modern hardware accelerators. Secondly, it performs similarly well for smaller dimensions of the K matrix, dk, as the additive attention. For larger dk, the scaled dot-product attention performs a bit worse because dot products can cause the vanishing gradient problem. This is compensated via the scaling factor, which is defined as [symbol] .

As discussed above, the attention function takes as input three objects: key, value, and query. In the context of Transformers, these objects are matrices of shapes (n, d), where n is the number of elements in the input sequence and d is the hidden representation of each element (also called the hidden vector). Attention is then computed as:

$$\text{Attention} \left(\text{Q, K, V}\right) = \text{SoftMax} \left(\frac{Q {K}^{T}}{\sqrt{d k}}\right) V$$

where Q, K, V are computed as:

$$X ⋅ {W}_{Q} , X ⋅ {W}_{K} , X ⋅ {W}_{V}$$

X is the input matrix and W_{Q}, W_{K}, W_{V} are learned weights to project the input matrix into the representations. The dot products appearing in the attention function are exploited for their geometrical interpretation where higher values of their results mean that the inputs are more similar, i.e., pointing in the geometrical space into the same direction. Since the attention function now works with matrices, the dot product becomes matrix multiplication. The SoftMax function is used to normalize the attention weights into the value of 1 prior to being multiplied by the values matrix. The resulting matrix is used either as input into another layer of attention or becomes the output of the Transformer.

### Multi-Head Attention

Transformers become even more powerful when multi-head attention is used. Queries, keys, and values are computed the same way as above, though they are now projected into h different representations of smaller dimensions using a set of h learned weights. Each representation is passed into a different scaled dot-product attention block called a head. The head then computes its output using the same procedure as described above.

Formally, the multi-head attention is defined as

$$\text{MultiHeadAttention} \left(\text{Q, K, V}\right) = \left[{\text{head}}_{1} , … , {\text{head}}_{\text{h}}\right] {\text{W}}_{0}$$

$${\text{where head}}_{\text{i}} = \text{Attention} \left(Q {W}_{i}^{Q} , K {W}_{i}^{K} , V {W}_{i}^{V}\right)$$

The outputs of all heads are concatenated together and projected again using the learned weights matrix W0 to match the dimensions expected by the next block of heads or the output of the Transformer. Using the multi-head attention instead of the simpler scaled dot-product attention enables Transformers to jointly attend to information from different representation subspaces at different positions.

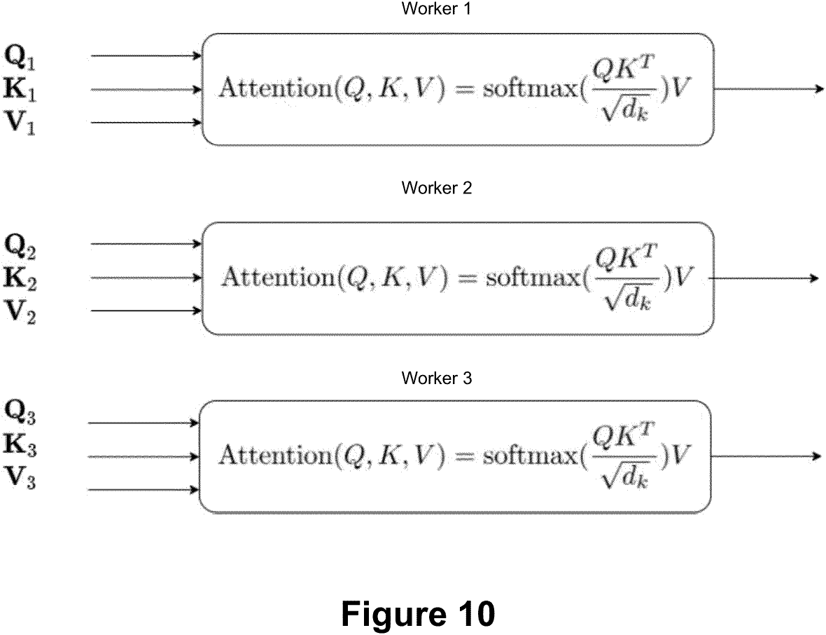

As shown in FIG. 10, one can use multiple workers to compute the multi-head attention in parallel, as the respective heads compute their outputs independently of one another. Parallel processing is one of the advantages of Transformers over RNNs.

Assuming the naive matrix multiplication algorithm which has a complexity of:

$$a ⋅ b ⋅ c$$

For matrices of shape (a, b) and (c, d), to obtain values Q, K, V, we need to compute the operations:

$$\text{X} ⋅ {\text{W}}_{\text{Q,}} \text{X} ⋅ {\text{W}}_{\text{K,}} \text{X} ⋅ \text{WV}$$

The matrix X is of shape (n, d) where n is the number of patches and d is the hidden vector dimension. The weights W_{Q}, W_{K}, W_{V} are all of shape (d, d). Omitting the constant factor 3, the resulting complexity is:

$$\text{n} ⋅ {\text{d}}^{2}$$

We can proceed to the estimation of the complexity of the attention function itself, i.e., of SoftMax

$$\left(\frac{Q {K}^{T}}{\sqrt{d k}}\right) V .$$

The matrices Q and K are both of shape (n, d). The transposition operation does not influence the asymptotic complexity of computing the dot product of matrices of shapes (n, d) • (d, n), therefore its complexity is:

$${\text{n}}^{2} ⋅ \text{d}$$

Scaling by a constant factor

$$\sqrt{d k}$$

where dk is the dimension of the keys vector, as well as applying the SoftMax function, both have the complexity of a • b for a matrix of shape (a, b), hence they do not influence the asymptotic complexity. Lastly the dot product SoftMax

$$\left(\frac{Q {K}^{T}}{\sqrt{d k}}\right) ⋅ V$$

is between matrices of shapes (n, n) and (n, d) and so its complexity is:

$${\text{n}}^{2} ⋅ \text{d}$$

The final asymptotic complexity of scaled dot-product attention is obtained by summing the complexities of computing Q, K, V, and of the attention function

$$\text{n} ⋅ {\text{d}}^{2} + {\text{n}}^{2} ⋅ \text{d} \text{.}$$

The asymptotic complexity of multi-head attention is the same since the original input matrix X is projected into h matrices of shapes

$$\left(\text{n,} \frac{d}{h}\right)$$

where h is the number of heads. From the view of asymptotic complexity, h is constant, therefore we would arrive at the same estimate of asymptotic complexity using a similar approach as for the scaled dot-product attention.

Transformer models often have the encoder-decoder architecture, although this is not necessarily the case. The encoder is built out of different encoder layers which are all constructed in the same way. The positional encodings are added to the embedding vectors. Afterward, self-attention is performed.

### Encoder Block of Transformer

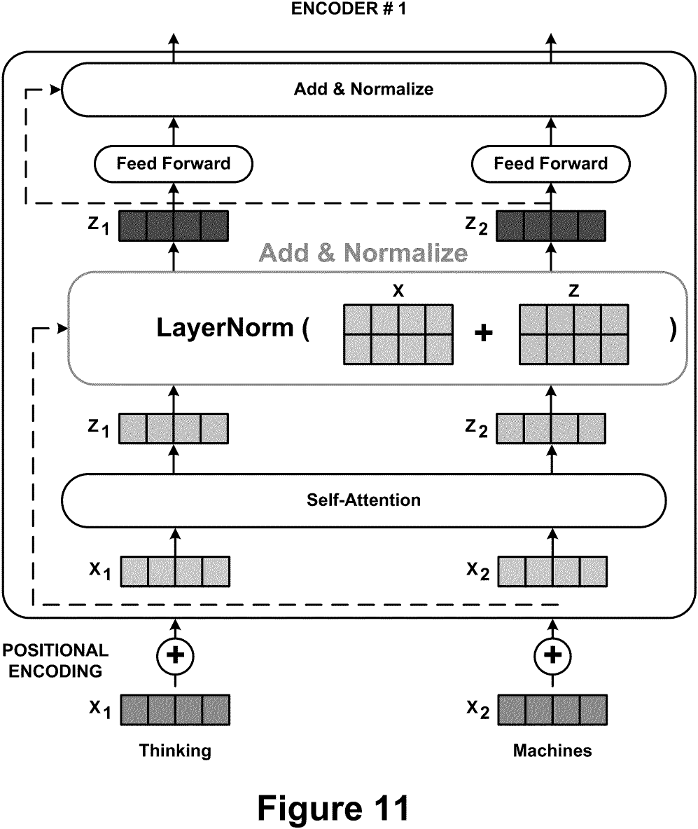

FIG. 11 portrays one encoder layer of a Transformer network. Every self-attention layer is surrounded by a residual connection, summing up the output and input of the self-attention. This sum is normalized, and the normalized vectors are fed to a feed-forward layer. Every z vector is fed separately to this feed-forward layer. The feed-forward layer is wrapped in a residual connection and the outcome is normalized too. Often, numerous encoder layers are piled to form the encoder. The output of the encoder is a fixed-size vector for every element of the input sequence.

Just like the encoder, the decoder is built from different decoder layers. In the decoder, a modified version of self-attention takes place. The query vector is only compared to the keys of previous output sequence elements. The elements further in the sequence are not known yet, as they still must be predicted. No information about these output elements may be used.

### Encoder-Decoder Blocks of Transformer

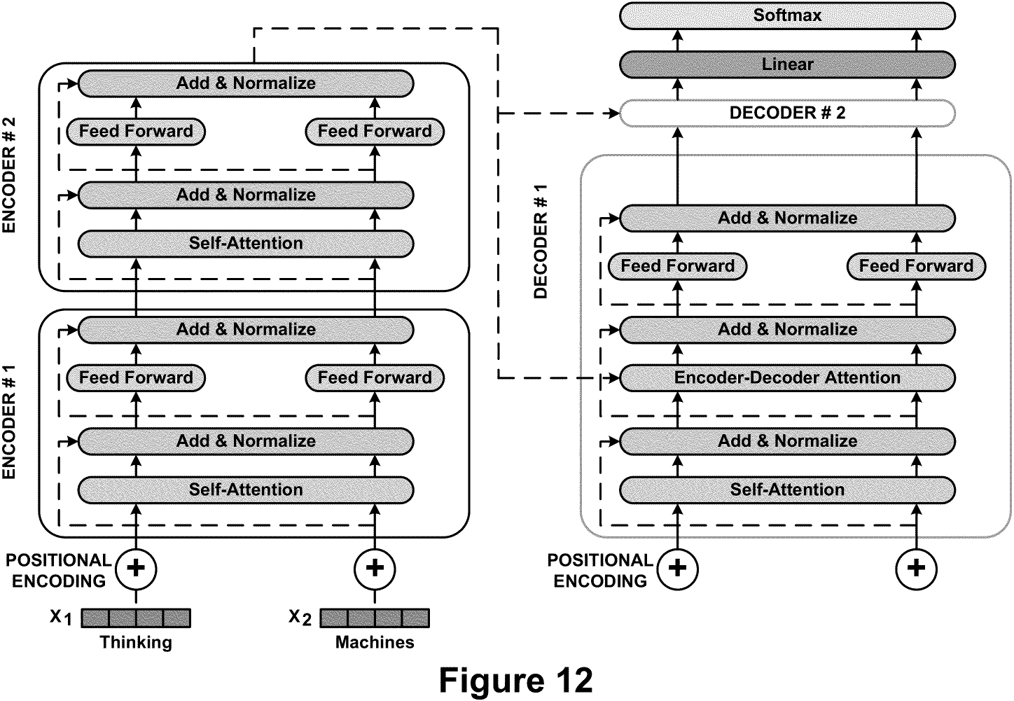

FIG. 12 shows a schematic overview of a Transformer model. Next to a self-attention layer, a layer of encoder-decoder attention is present in the decoder, in which the decoder can examine the last Z vectors of the encoder, providing fluent information transmission. The ultimate decoder layer is a feed-forward layer. All layers are packed in a residual connection. This allows the decoder to examine all previously predicted outputs and all encoded input vectors to predict the next output. Thus, information from the encoder is provided to the decoder, which could improve the predictive capacity. The output vectors of the last decoder layer need to be processed to form the output of the entire system. This is done by a combination of a feed-forward layer and a SoftMax function. The output corresponding to the highest probability is the predicted output value for a subject time step.

For some tasks other than translation, only an encoder is needed. This is true for both document classification and name entity recognition. In these cases, the encoded input vectors are the input of the feed-forward layer and the SoftMax layer. These models have applications in the field of biology as well for predicting protein structure and function and labeling DNA sequences.

### Vision Transformer

There are extensive applications of transformers in vision including popular recognition tasks (e.g., image classification, object detection, action recognition, and segmentation), generative modeling, multi-modal tasks (e.g., visual-question answering, visual reasoning, and visual grounding), video processing (e.g., activity recognition, video forecasting), low-level vision (e.g., image super-resolution, image enhancement, and colorization) and 3D analysis (e.g., point cloud classification and segmentation).

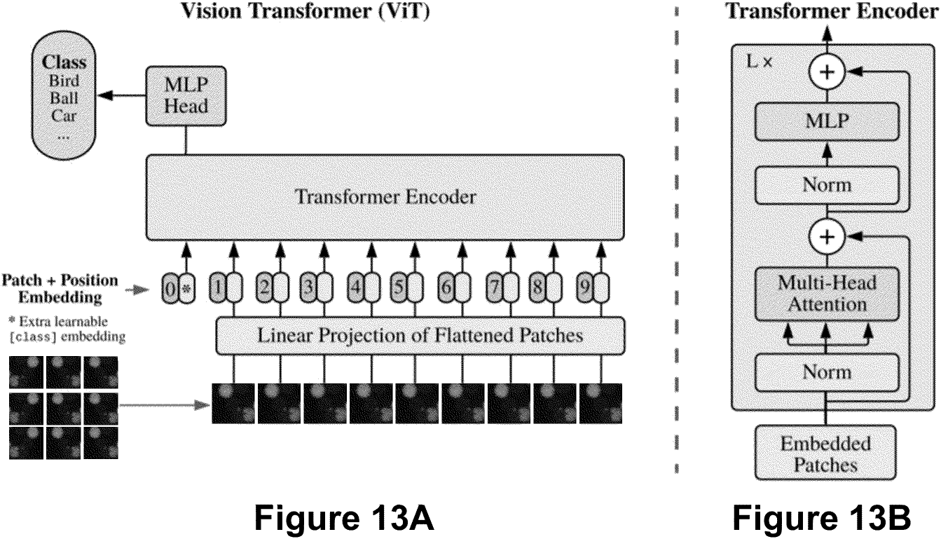

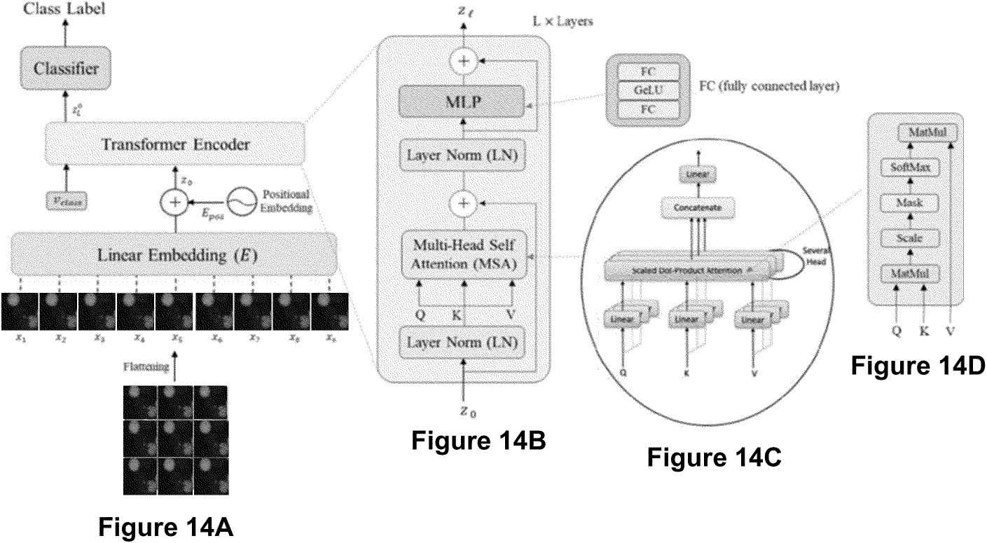

In image classification, we often have a single input image in which the pixels are in a sequence. To reduce the computation required, Vision Transformers (ViTs) cut the input image into a set of fixed-sized patches of pixels. The patches are often 16 × 16 pixels. ViTs are depicted in FIGS. 13A, 13B, 14A, 14B, 14C, and 14D. Unfortunately, important positional information is lost because image sets are position-invariant. This problem is solved by adding a learned positional encoding into the image patches.

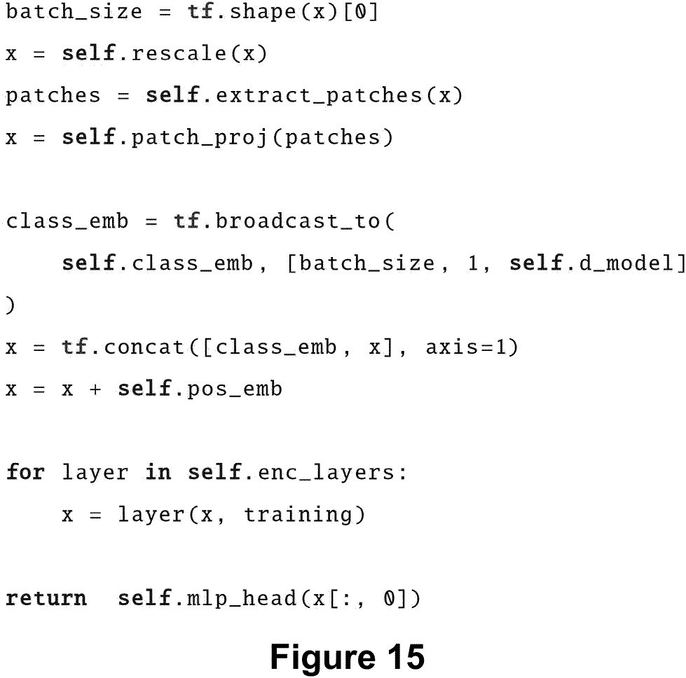

The computations of the ViT architecture can be summarized as follows. The first layer of a ViT extracts a fixed number of patches from an input image (FIG. 13A). The patches are then projected to linear embeddings. A special class token vector is added to the sequence of embedding vectors to include all representative information of all tokens through the multi-layer encoding procedure. The class vector is unique to each image. Vectors containing positional information are combined with the embeddings and the class token. The sequence of embedding vectors is passed into the Transformer blocks. The class token vector is extracted from the output of the last Transformer block and is passed into a multilayer perceptron (MLP) head whose output is the final classification. The perceptron takes the normalized input and places the output in categories. It classifies the images. This procedure directly translates into the Python Keras code shown in FIG. 15.

When the input image is split into patches, a fixed patch size is specified before instantiating a ViT. Given the quadratic complexity of attention, patch size has a large effect on the length of training and inference time. A single Transformer block comprises several layers. The first layer implements Layer Normalization, followed by the multi-head attention that is responsible for the performance of ViTs. In the depiction of a Transformer block in FIG. 13B, we can see two arrows. These are residual skip connections. Including skip connection data can simplify the output and improve the results. The output of the multi-head attention is followed again by Layer Normalization. And finally, the output layer is an MLP (Multi-Layer Perceptron) with the GELU (Gaussian Error Linear Unit) activation function.

ViTs can be pretrained and fine-tuned. Pretraining is generally done on a large dataset. Fine-tuning is done on a domain specific dataset.

Domain-specific architectures, like convolutional neural networks (CNNs) or long short-term memory networks (LSTMs), have been derived from the usual architecture of MLPs and suffer from so-called inductive biases that predispose the networks towards a certain output. ViTs stepped in the opposite directions of CNNs and LSTMs and became more general architectures by eliminating inductive biases. A ViT can be seen as a generalization of MLPs because MLPs, after being trained, do not change their weights for different inputs. On the other hand, ViTs compute their attention weights at runtime based on the particular input.

### Transformer Models as Applied to Genomics

The following discussion describes some implementations of how the Transformer models process a genomic sequence and produce position-wise nucleotide classification of the genomic sequence.

The Transformer models include convolutional layers that can detect local patterns, and thereby enhance the detection of nucleotide motifs. The Transformer models process a genome sequence in consecutive segments of length l. Every input nucleotide x E {A, C, G, T} is first transformed into a vector embedding h(0), after which it is transformed k times through addition (residual connection) with another vector, obtained by the multi-head attention function present in each layer (h(0) → ... → h(k)).

A set of fully connected layers transforms h(k) into a model output y(k). For each residual block, the vector that is summed with the input (to obtain h(1), ... , h(k)) is calculated using the hidden states of l upstream positions.

The multi-head attention applied in each residual block is methodologically identical. From each input hidden state h, a query (q), key (k), and value (v) vector of equal shapes are calculated. The output z of the attention head, applied on the hidden state at position n, is calculated as follows:

$${\text{z}}^{\left(\text{n}\right)} = \text{softmax} \left(\frac{{\text{q}}^{\left(\text{n}\right)} \text{.K}}{\sqrt{{\text{d}}_{\text{head}}}}\right) . \text{V,}$$

where K, V ∈ ℝ^{1×d_{heads}} are the matrices that are composed from l upstream hidden states (e.g., K =[k⁽ⁿ⁻¹⁾, . . . , k⁽ⁿ⁾]).

The denominator is used to stabilize the scores based on the dimensions of q, k, and v (d_{head}). The multiplication of the query vector with all the key vectors results in a vector of scores that is normalized for all input values using the softmax function. These scores are multiplied to the v vectors for the calculation of z (i.e., a linear combination). The attention scores denote the relevance of information present between two positions, where the multiplication of the q and k vectors function as a lock and key encoding, which returns goodness-of-fit scores for the information embedded in two hidden states (defined by v).

In each residual block, multiple attention heads are present (hence, multi-head attention), each featuring their own unique sets of model weights to calculate q, k, and v. As such, multiple types of information can be extracted from the input hidden states. The outcome of different attention heads within the same layer is further processed into a single vector, which is summed with h to obtain the hidden state of the next layer (e.g., h(1) → h(2)).

Contextual information embedded within the hidden states derived from single nucleotides is limited. Motifs formed from multiple neighboring nucleotides are deemed of greater importance towards biological processes. The addition of a convolutional layer allows the q, k, and v vectors to be derived from multiple neighboring hidden states without affecting the input/output resolution. Thereby, the retrieval of relevant information using attention is improved, resulting in improved predictive performances on a variety of tasks.

Positional information is used within the vectors q, k, and v by superimposing (i.e., through summation) a positional encoding vector to h. The added signal is a function of the vector index and the relative positioning with respect to the other input hidden states.

The annotation of DNA is a sequence labeling task that has correspondences in natural language processing. The DNA sequence is a data set of n nucleotides, i.e., X ∈ {x(1), x(2), ..., x(n)}, where x ∈ A, C, T, G, the task comprises predicting a label y 0, 1 for each position x, where a positive label denotes the occurrence of an event at that position.

The Transformer models process the genome in sequential segments of l nucleotides. During training, a non-linear transformation function E is optimized that maps the input classes {A, C, T, G} to a vector embedding h of length d_{model}. For nucleotide x(i) on the genome:

$$\text{h=E} \left({\text{x}}^{\left(\text{i}\right)}\right) , {\text{x}}^{\left(\text{i}\right)} ∈ \left\{\text{A,T,C,G}\right\} ,$$

$$\text{where} h ∈ {ℝ}^{{d}_{m o d e l}} .$$

The hidden states of each segment H ∈ ℝ^{lxd}model, [h⁽¹⁾, ..., h^{(l)}], are processed through k layers. As such, the data propagation through the network for any input x follows multiple transformation: x → h^{(0,:)} →... → h^{(k,:)} → ŷ.

Within each layer, multi-head attention is calculated for each hidden state. Next, for each hidden state of h, the output of the multi-head attention step (MultiHead) is summed with the input, i.e., a residual connection, with the final step being layer normalization. The calculations of the output for all hidden states h in layer t at position m of segment s are performed in parallel:

$${\text{h}}^{\left(\text{s,t+l,m}\right)} = \text{LayerNorm} \left({\text{h}}^{\left(\text{s,t,m}\right)} + \text{MultiHead} \left({\text{H}}^{\left(\text{s,t}\right)}\right)\right) ,$$

or

$${\text{H}}^{\left(\text{s,t+l}\right)} = \text{LayerNorm} \left({\text{H}}^{\left(\text{s,t}\right)} + \text{MultiHead} \left({\text{H}}^{\left(\text{s,t}\right)}\right)\right) ,$$

$$\text{where t} ∈ \left[0 , \text{k} \left[\text{and m} ∈\right)\right) \left[1 , 1\right] .$$

After a forward pass through k layers, a final linear combination reduces the dimension of the output hidden state (d_{model}) to the amount of output classes. In one implementation, only binary classification is performed. In another implementation, a softmax layer is applied before obtaining the prediction value ŷᵢ for nucleotide xᵢ.

### Particular Implementation of Using Second Model to Predict Variant Pathogenicity

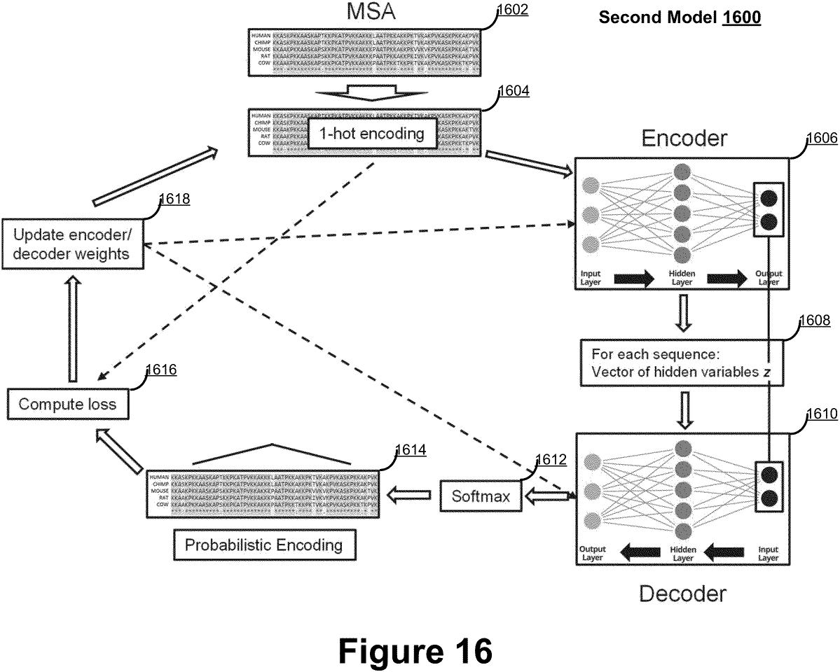

An example of using variational autoencoder (VAE) as the second model to predict pathogenicity scores of variants will be described in more detail in accordance with FIG. 16. Here, the second model is a VAE-based non-linear latent-variable model that captures high-order epistasis in proteins. The non-linear latent variable model posits latent (hidden) variables z that can jointly influence many positions at the same time. The dependency p(xlz) of the sequence x on the latent variables z is modeled by a neural network. The inference and learning are made tractable by jointly training with an approximate inference network q(zlx).

In particular, the second model is used to implicitly capture higher-order interactions between positions in a sequence in a protein family. For every observed sequence x, unobserved latent variables z together with a generative process p(z)p(x|z) will be generated that specifies a joint distribution over hidden variables and observed variables. As the marginal probability of the observed data, p(x), is difficult to directly compute because it is intractable, variational inference is used here to form a lower bound, namely, Evidence Lower Bound (ELBO), on the log probability. ELBO used in this model takes the form of:

$$log p \left(x \vert θ\right) \geq E \text{q} \left[log p \left(x \vert z , θ\right)\right] - {D}_{K L} \left(q \left(z \vert x , φ\right) \left‖p \left(z\right)\right)\right) ,$$

where q(z|x) is an approximate posterior for hidden variables given the observed variables p(z|x).

Thus, given a starting target wild-type sequence, the pathogenicity effect caused by a single point mutation can be computed as the difference in ELBOs and predicted as:

$$log \frac{p \left(\text{xmutant}\right)}{p \left(\text{xwildtype}\right)}$$

As illustrated in FIG. 16, a target wild-type sequence (Swt) and a plurality of homologous non-target sequences aligned in MSA are used as input of the model (step 1602), each sequence being one-hot-encoded (step 1604). In one implementation, for a target sequence, MSA of the corresponding protein family may be obtained by five search iterations of the profile HMM homology search tool j ackhmmer against the UniRef100 database of non-redundant protein sequences (released in November 2015). See, Eddy, S.R., Accelerated Profile HMM Searches.PLoS Comput Biol, 2011. 7(10): p. e1002195; Suzek, B.E., et al., UniRef clusters: a comprehensive and scalable alternative for improving sequence similarity searches. Bioinformatics, 2015. 31(6): p. 926-32.

The target wild-type sequence (Swt) is fed into an encoder and compressed to a vector of hidden variables z (steps 1606 and 1608). In one implementation, the encoder has an architecture of 1500-1500-30 with fully connected layers and ReLU non-linearity functions (See Exhibit 7). The vector of hidden variables z is further decoded via a decoder and thus, generates a reconstructed sequence

$$\hat{S w t}$$

. In one implementation, the decoder has two hidden layers: the first with size 100 and a ReLU non-linearity, and the second with size 2,000 with a sigmoid non-linearity (See Exhibit 7). Thus, p(x_{wildtype}) can be calculated as:

$$p \left({\text{x}}_{\text{wildtype}}\right) = p \left(S w t \vert \hat{S w t}\right)$$

With softmax (step 1612) and probabilistic encoding (step 1614), the computed loss via ELBO as described above (step 1616) is used to update the weights in the encoder and decoder (step 1618).

When a single point mutation is introduced, the steps from 1602 to 1618 are repeated. In other words, an alternative protein sequence with a single point mutation (Smut) is encoded and compressed to a vector of hidden variables z. The vector of hidden variables z is further decoded via the decoder and generates a reconstructed sequence

$$\hat{S m u t} .$$

. Thus, p(xₘᵤₜₐₙₜ) can be calculated as:

$$\text{p} \left({\text{x}}_{\text{mutant}}\right) = \text{p} \left(\text{Smut\textbar} \hat{S m u t}\right)$$

The effect of the single point mutation (e.g., pathogenicity) can be quantified as the log ratio of mutant likelihood to wild-type likelihood as approximated by the ELBO:

$$log \frac{p \left(\text{xmutant}\right)}{p \left(\text{xwildtype}\right)}$$

As described above, this non-linear latent variable model learns in an unsupervised manner solely from sequence information from large multiple sequence alignments. It captures beyond-pairwise features in high-order epistasis, despite the model requiring high computation power (e.g., four hours per protein on GPU) and input sequence alignment to be as deep as possible.

### Inter-model Variant Pathogenicity Score Reassignment

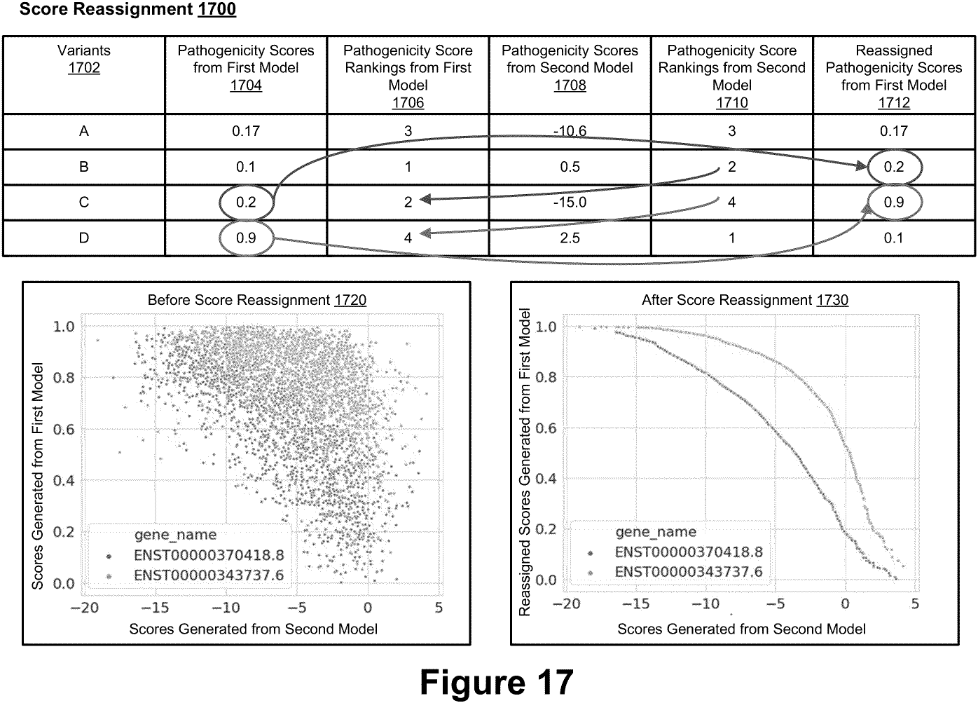

FIG. 17 illustrates an example of inter-model variant pathogenicity score reassignment 1700. As illustrated in the table, a set of target variants 1702 indicated by A, B, C, and D, respectively, are used by the two models to predict their corresponding pathogenicity score. As described above in, e.g., FIGS. 1, 2 and 5, the first model and the second model independently predict the pathogenicity of each variant and generate a pathogenicity score-and-ranking mapping. For each variant, the first model generates its corresponding pathogenicity score-and-ranking mapping that includes a pathogenicity score 1704 mapped to a pathogenicity score ranking 1706. Similarly, for each variant, the second model generates its corresponding pathogenicity score-and-ranking mapping that includes a pathogenicity score 1708 mapped to a pathogenicity score ranking 1710.

The same variant may have different values and scales in pathogenicity scores from the two models. For example, variant A has a pathogenicity score of 0.7 and a ranking of 3 generated from the first model, yet a score of -10.6 and a ranking of 3 from the second model. In another implementation, the same variant not only has different pathogenicity scores and but also different rankings. For example, variant D has a pathogenicity score of 0.9 and a ranking of 4 generated from the first model, yet a score of 2.5 and a ranking of 1 from the second model.

For each variant, a rule-based reassignment logic (e.g., the reassignment logic 108 including a ranking comparator 118 and a score reassignor 128 as described in FIG. 1) automatically compares the rankings and reassigns the pathogenicity scores. For example, the ranking comparator 118 compares the pathogenicity score ranking 1706 from the first model with the ranking 1710 from the second model. The score reassignor 128 reassigns a pathogenicity score to the variant based on the ranking of the same variant generated from the second model.

Consider variant C listed in the table of FIG. 17 as an example. Variant C has a pathogenicity score of 0.2 generated from the first model and a corresponding ranking of 2. Yet, the same variant has a score of -15.0 from the second model and a corresponding ranking of 4. The ranking comparator 118 compares the two rankings, namely, 2 and 4. The score reassignor 128 reassigns another pathogenicity score of 0.9 to variant C, based on the ranking of 4 generated from the second model. It is noted that the pathogenicity score of 0.9 was originally assigned to variant D with a ranking of 4 generated from the first model.

Consider variant B listed in the table of FIG. 17 as another example. It has a pathogenicity score of 0.1 generated from the first model and a corresponding ranking of 1. Yet, the same variant has a score of 0.5 and a corresponding ranking of 2 from the second model. The ranking comparator 118 compares the two rankings, namely, 1 and 2, generated from the two models. The score reassignor 128 reassigns another pathogenicity score of 0.2 to variant B, based on the ranking of 2 generated from the second model. It is noted that the pathogenicity score of 0.2 was originally assigned to variant C with a ranking of 2 generated from the first model.

As listed in columns 1704 and 1706 of the table, variants A, B, C and D originally have a given pathogenicity score of 0.7, 0.1, 0.2 and 0.9 generated from the first model, each score being mapped to a pathogenicity score ranking of 3, 1, 2 and 4, respectively. After the reassignment, as further listed in column 1712 of the table, variants A, B, C and D currently have reassigned pathogenicity score of 0.7, 0.2, 0.9 and 0.1, respectively, but each score is currently mapped to a pathogenicity score ranking of 3, 2, 4 and 1, respectively. In other words, the reassignment logic utilizes the ranking 1710 of each variant generated from the second model (i.e., variant A - ranking 3, variant B - ranking 2, variant C - ranking 4 and variant D - ranking 1) and reassigns a pathogenicity score to each variant based on this ranking. Meanwhile, the numerical values and scales of the reassigned pathogenicity scores remain the same.

The graphs 1720 and 1730 of FIG. 17 illustrate the correlation between two sets of pathogenicity scores generated from the two models before and after score reassignment. Two human genes samples are evaluated herein. ENST00000370418.8 represents Homo sapiens carboxypeptidase N subunit 1 (CPN1) and ENST00000343737.6 represents Homo sapiens Wnt family member 8B (WNT8B), respectively. Before the score reassignment as illustrated in graph 1720, both genes have scattered pathogenicity scores generated from the two models. And the two sets of scores generated from the two models substantially overlap with one another. After the reassignment as illustrated in graph 1730, both genes have a good correlation between the reassigned scores generated from the first model and the scores generated from the second model.

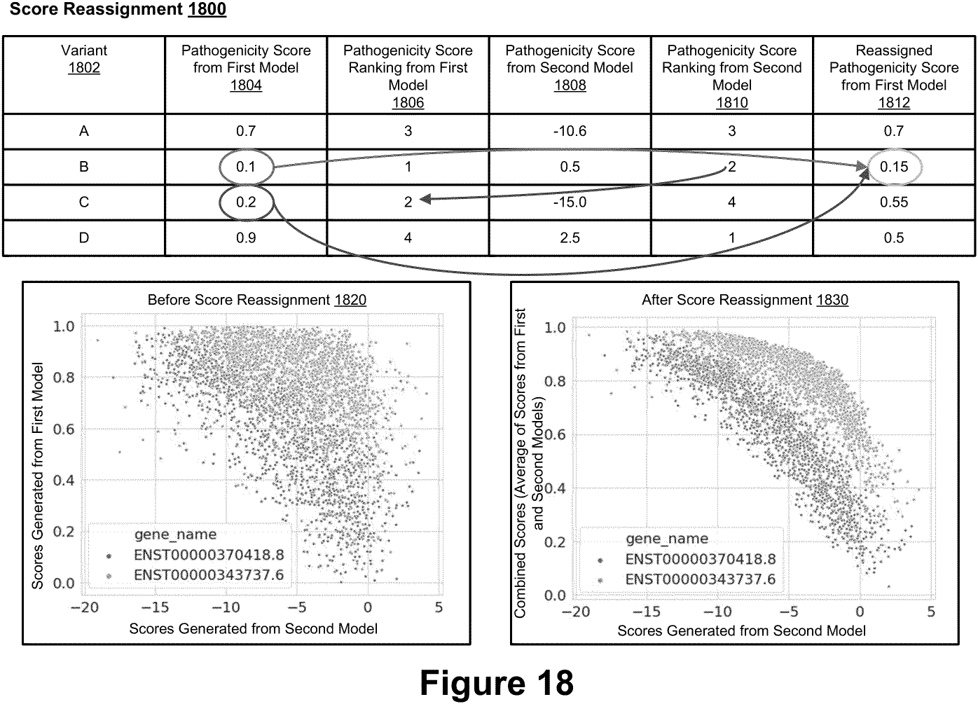

FIG. 18 illustrates another example of inter-model variant pathogenicity score reassignment 1800. Unlike the example illustrated in FIG. 17 where the reassigned pathogenicity score sets have the same numerical values and scales as the original scores, here, a new score set is calculated by combining both the original scores and the assigned scores. As illustrated in the table, a set of target variants 1802 indicated by A, B, C, and D, respectively, are used by the two models to predict their corresponding pathogenicity scores. As described above in, e.g., FIGS. 1 and 2, the first model and the second model independently predict the pathogenicity of each variant and generate a pathogenicity score-and-ranking mapping. For each variant, the first model generates a corresponding pathogenicity score-and-ranking mapping including a pathogenicity score 1804 mapped to a pathogenicity score ranking 1806. Similarly, the second model generates a pathogenicity score-and-ranking mapping including a pathogenicity score 1808 mapped to a pathogenicity score ranking 1810.

The rule-based reassignment logic (e.g., reassignment logic 108 including a ranking comparator 118 and a score reassignor 128 as described in FIG. 1) automatically compares the rankings and reassigns the pathogenicity scores. In one implementation, the reassignment logic 108 further includes a combination logic that automatically combines the originally assigned pathogenicity score and the reassigned score, to generate a combined pathogenicity score.

Consider variant B listed in the table of FIG. 18 as an example. Variant B has a pathogenicity score of 0.1 and a ranking of 1 generated from the first model. The same variant has a score of 0.5 and a ranking of 2 from the second model. The ranking comparator 118 compares the two rankings, namely, 1 and 2. The score reassignor 128 reassigns the pathogenicity score of 0.2 to variant B, based on the ranking of 2 generated from the second model. The combination logic combines the originally assigned score of 0.1 and the newly reassigned score of 0.2, and generates a combined pathogenicity score. Here, the combined pathogenicity score of 0.15 for variant B is an average of the originally assigned score and the newly reassigned score.

As listed in columns 1804 and 1806 of the table, variants A, B, C and D originally have a given pathogenicity score of 0.7, 0.1, 0.2 and 0.9 generated from the first model, each score being mapped to a pathogenicity score ranking of 3, 1, 2 and 4, respectively. After the reassignment, as further listed in column 1812 of the table, variants A, B, C and D currently have reassigned pathogenicity score of 0.7, 0.15, 0.55 and 0.5, respectively. Each score is currently mapped to a new pathogenicity score ranking of 4, 1, 3 and 2, respectively. In other words, the reassignment logic utilizes the ranking 1810 of each variant generated from the second model (i.e., variant A - ranking 3, variant B - ranking 2, variant C - ranking 4 and variant D - ranking 1) and reassigns a pathogenicity score to each variant based on this ranking. Unlike column 1712 of the table in FIG. 17 where the reassigned scores have the same numerical values and scales as the original scores listed in column 1704, here, the combined scores listed in column 1812 is different in numerical values from the original scores listed in column 1804. Additionally, each combined score will be mapped to a new ranking as output. For example, variants A, B, C and D now have the new ranking of 4, 1, 3 and 2, respectively, based on their combined scores listed in column 1812. This new ranking can be different from both the original rankings generated from the first model and the second model as listed in columns 1806 and 1810.

In another implementation, the combined pathogenicity score is a weighted average of the originally assigned score and the reassigned score. The weights used for the weighted average may be preset and specified for the first model and the second model, respectively. In another implementation, the weights used for the weighted average may correspond to the respective ranking of the originally assigned score and the reassigned score.

In yet another implementation, the combined pathogenicity score is a sum of the originally assigned score and the reassigned score. In yet another implementation, the combined pathogenicity score is a weighted sum of the originally assigned score and the reassigned score. The weights used for the weighted sum may be preset and specified for the first model and the second model, respectively. In another implementation, the weights used for the weighted sum may correspond to the respective ranking of the originally assigned score and the reassigned score.

The graphs 1820 and 1830 of FIG. 18 illustrate the correlation between two sets of pathogenicity scores generated from the two models before and after score reassignment. Here, y-axis of graph 1830 represents combined scores by averaging originally assigned scores generated from the first model and the reassigned scores based on the rankings generated the second model, in consistence with the table in FIG. 18. Two human genes samples (ENST00000370418.8 and ENST00000343737.6) are evaluated herein. Before the score reassignment as illustrated in graph 1820, both genes have scattered pathogenicity scores generated from the two models. And the two sets of scores generated from the two models substantially overlap with one another. After the reassignment as illustrated in graph 1830, both genes have a good correlation between the combined scores generated from the first model and the scores generated from the second model.

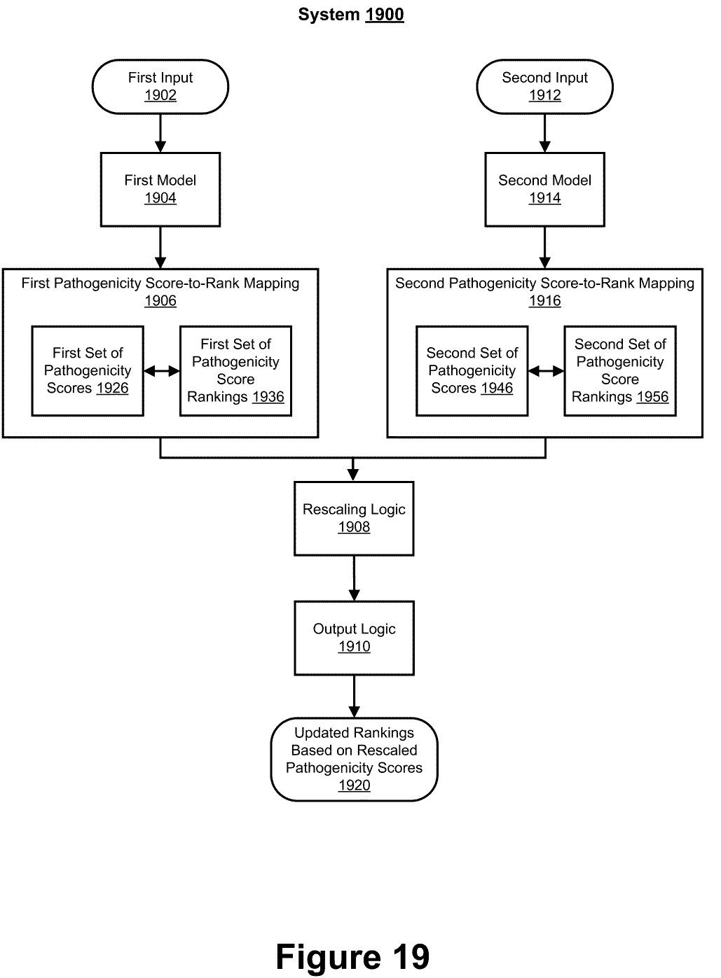

FIG. 19 illustrates another example system including two models for generating and pathogenicity score-to-rank mapping for a target variant and recalibrating pathogenicity scores. As illustrated, the example system 1900 includes two models for generating pathogenicity score-to-rank mappings for target variants and reassigning pathogenicity scores. First model 1904, based on first input 1902, generates a first pathogenicity score-to-rank mapping 1906, where the mapping includes a first set of pathogenicity scores 1926 and a first set of pathogenicity score rankings 1936. The first input 1902 can be similar to the first input 102 illustrated in FIG. 1.

In one implementation, the first model 1904 uses a first scale of pathogenicity scores, for example, ranging from 0 to 1, to differentiate pathogenic variants from benign variants. That is, each score within the first set of pathogenicity scores 126 may range from 0 to 1, where a lower score indicates a benign variant accompanied by a higher ranking while a higher score indicates a pathogenic variant accompanied by a lower ranking.

Second model 1914, based on second input 1912, generates a second pathogenicity score-to-rank mapping 1916, where the mapping includes a second set of pathogenicity scores 1946 and a second set of pathogenicity score rankings 1956. The second input 1912 can be similar to the second input 112 illustrated in FIG. 1.

In one implementation, the second model 1914 uses a second scale of pathogenicity scores, for example, ranging from a maximum real number represented digitally to a minimum real number represented digitally, to differentiate pathogenic variants from benign variants. That is, each score within the second set of pathogenicity scores 1946 may range anywhere from -∞ to +∞, where a lower score indicates a pathogenic variant accompanied by a lower ranking within the second set of pathogenicity score rankings 1956, while a higher score indicates a benign variant accompanied by a lower ranking.

Thus, for the same set of variants, the first model 1904 generates a first set of pathogenicity scores 1926 on a first scale, while the second model 1914 generates a second set of pathogenicity scores 1946 on a second scale different from the first scale. Accordingly, the system 1900 includes a rescaling logic 1908 that rescales the second set of pathogenicity scores 1946. Alternatively, the rescaling logic 1908 can rescale the first set of pathogenicity scores 1926. In one implementation, the rescaling algorithms may be preset and output logic 1910 may output an updated ranking of the set of variants 1920, based on their rescaled pathogenicity scores.

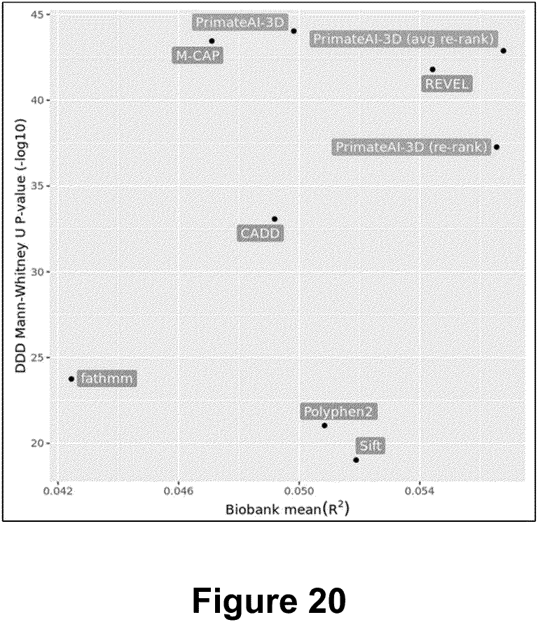

The performance of the disclosed implementations of inter-model re-ranking system is evaluated, along with other popular classification algorithms (Sift, Polyphen2, fathmm, CADD, M-CAP and REVEL). The first model used in the inter-model re-ranking system is PrimateAI 3D, and the second model is a VAE-based non-linear latent variable model. As illustrated in FIG. 20, “PrimateAI-3D” refers to the first model as benchmark. “PrimateAI-3D (re-rank)” refers to an inter-model re-ranking system, where the system reassigns the pathogenicity scores generated from PrimateAI-3D (i.e., first model) to the variants based on the pathogenicity score rankings generated from VAE-based non-linear latent variable model (i.e., second model). “PrimateAI-3D (avg re-rank)” refers another inter-model re-ranking system, where the system reassigns the pathogenicity scores generated from PrimateAI-3D (i.e., first model) to the variants based on the pathogenicity score rankings generated from VAE-based non-linear latent variable model (i.e., second model).

Two different datasets are used for the evaluation. One dataset includes rare variants from UK Biobank found in genes, and these variants are significantly associated with certain quantitative phenotypes, e.g., LDL cholesterol level. The disclosed implementation of inter-model re-ranking system is used to predict the quantitative phenotypes of human genes from the rare variants found in the genes, along with other classification algorithms. The p-values are generated from Spearman rank correlation.

Another dataset includes rare variants from patients with developmental delay (DDD) or autism (ASD), together with rare variants from healthy controls. The disclosed implementation of inter-model re-ranking system is used to distinguish rare variants found in the patients with DDD or ASD from those found in controls. The p-values are generated from Wilcoxon ranksum test. As illustrated in FIG. 10, y-axis indicates DDD Mann-Whitney U P-value (-log10).

The disclosed system for inter-model pathogenicity score recalibration has at least the following advantages. By taking into the considerations pathogenicity scores and rankings generated from different models, reassigning the scores and updating the rankings, the disclosed system improves the accuracy in predicting variant pathogenicity and assess the effects of variants on phenotypes. More importantly, such prediction accounts for not only single-position or pairwise mutations, but also for higher-order epistasis.

As we described above in various implementations, some deep learning models can be site-independent or pairwise interaction models that focus on single-position mutation or pairwise mutations at proximate positions. These models use structural information of proteins and evolutionary profiles from human and primate, and are trained primarily using common human variants and common non-human primate variants that are mapped to human variants. As a result, these models outperform other models that solely rely on sequence information, because the scale of the pathogenicity scores across protein domains and proteins is consistent and optimal. However, the predictions of these site-independent or pairwise interaction models do not consider high-order epistasis.

On the other hand, some deep learning models account for the effects of variants in a surrounding context and thus, are strong tools in modeling epistasis in proteins. An example mentioned above is protein language models that identify the context that confers meaning to each position in a sequence. These models can have hundreds of millions of parameters in the model architecture and be trained on hundreds of millions of diverse protein sequences across evolution. See, Language models enable zero-shot prediction of the effects of mutations on protein function, Joshua Meier, Roshan Rao, Robert Verkuil, Jason Liu, Tom Sercu, Alexander Rives, BioRxiv, doi: https://doi.org/10.1101/2021.07.09.450648. These models solely use a large and diverse database in MSA as input and thus, the ranking of the pathogenicity scores within a protein domain is optimal, despite the scale of the scores may not precisely reflect the pathogenicity across domains or proteins.

The disclosed technology for inter-model pathogenicity score reassignment provides an approach of combining the strengths of various models. As such, the rankings of the pathogenicity scores within a protein domain generated from one model (e.g., generative model, protein language model) accurately reflects the effects of the variants in the surrounding context (i.e., epistasis). Furthermore, the scale of scores (e.g., discriminative model) across different protein domains and different proteins remains optimal and consistent. It avoids undesired situations where the same pathogenicity score indicates high pathogenicity in one protein domain but entirely benign in another.

### Computer System

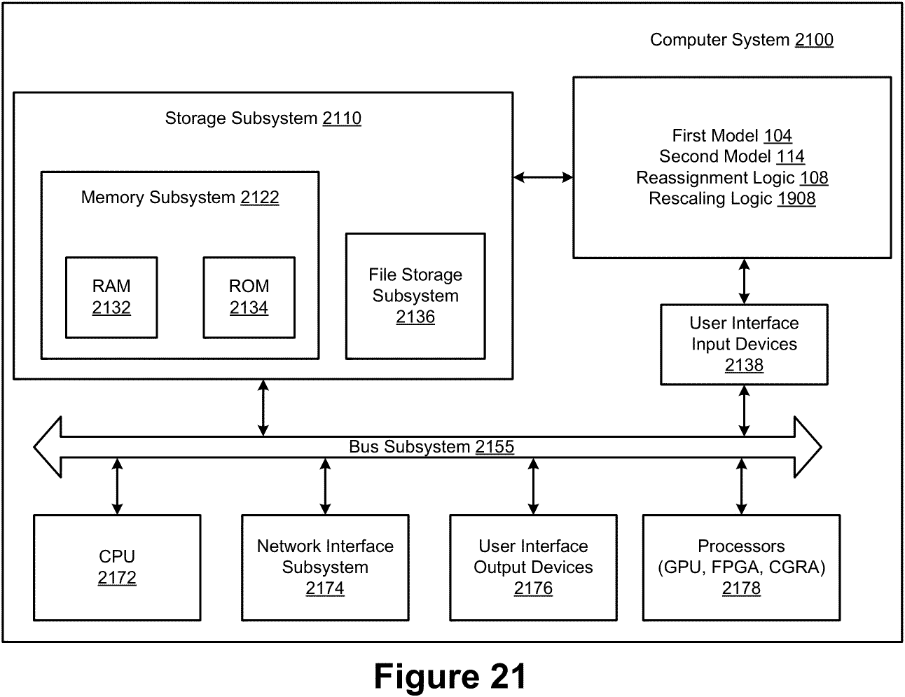

FIG. 21 is an example computer system 2100 that can be used to implement the technology disclosed. Computer system 2100 includes at least one central processing unit (CPU) 2172 that communicates with a number of peripheral devices via bus subsystem 2155. These peripheral devices can include a storage subsystem 2110 including, for example, memory devices and a file storage subsystem 2136, user interface input devices 2138, user interface output devices 2176, and a network interface subsystem 2174. The input and output devices allow user interaction with computer system 2100. Network interface subsystem 2174 provides an interface to outside networks, including an interface to corresponding interface devices in other computer systems.

In one implementation, the first and second models, including the pathogenicity classifier, are communicably linked to the storage subsystem 2110 and the user interface input devices 2138. In other implementations, the reassignment logic and the rescaling logic can also be communicably linked to the storage subsystem 2110 and the user interface input devices 2138.

User interface input devices 2138 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 2100.

User interface output devices 2176 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 2100 to the user or to another machine or computer system.

Storage subsystem 2110 stores programming and data constructs that provide the functionality of some or all of the modules and methods described herein. These software modules are generally executed by processors 2178.

Processors 2178 can be graphics processing units (GPUs), field-programmable gate arrays (FPGAs), application-specific integrated circuits (ASICs), and/or coarse-grained reconfigurable architectures (CGRAs). Processors 2178 can be hosted by a deep learning cloud platform such as Google Cloud Platform™, Xilinx™, and Cirrascale™. Examples of processors 2178 include Google’s Tensor Processing Unit (TPU)™, rackmount solutions like GX4 Rackmount Series™, GX36 Rackmount Series™, NVIDIA DGX-1™, Microsoft’ Stratix V FPGA™, Graphcore’s Intelligent Processor Unit (IPU)™, Qualcomm’s Zeroth Platform™ with Snapdragon processors™, NVIDIA’s Volta™, NVIDIA’s DRIVE PX™, NVIDIA’s JETSON TX1/TX2 MODULE™, Intel’s Nirvana™, Movidius VPU™, Fujitsu DPI™, ARM’s DynamicIQ™, IBM TrueNorth™, Lambda GPU Server with Testa V 100s™, and others.

Memory subsystem 2122 used in the storage subsystem 2110 can include a number of memories including a main random access memory (RAM) 2132 for storage of instructions and data during program execution and a read only memory (ROM) 2134 in which fixed instructions are stored. A file storage subsystem 2136 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 2136 in the storage subsystem 2110, or in other machines accessible by the processor.

Bus subsystem 2155 provides a mechanism for letting the various components and subsystems of computer system 2100 communicate with each other as intended. Although bus subsystem 2155 is shown schematically as a single bus, alternative implementations of the bus subsystem can use multiple busses.

Computer system 2100 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 2100 depicted in FIG. 21 is intended only as a specific example for purposes of illustrating the preferred implementations of the present invention. Many other configurations of computer system 2100 are possible having more or less components than the computer system depicted in FIG. 21.

### CLAUSES

The technology disclosed, in particularly, the clauses disclosed in this section, can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections -these recitations are hereby incorporated forward by reference into each of the following implementations.

One or more implementations and clauses of the technology disclosed, or elements thereof can be implemented in the form of a computer product, including a non-transitory computer readable storage medium with computer usable program code for performing the method steps indicated. Furthermore, one or more implementations and clauses of the technology disclosed, or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

### Clause Set 1

1. A system, comprising:
a first model (e.g., PrimateAI, PrimateAI 3D, JigsawAI) configured to generate, based in part on evolutionary conservation summary statistics [evolutionary profiles - examples: PSFM and PSSM] of amino acids in a target protein sequence,
   a first score-to-rank mapping that maps a first set of pathogenicity scores for a set of variants observed in the target protein sequence to a first set of score rankings;
a second model (e.g., DeepSequence in Exhibit 7, Transformer-based models (e.g., the one shown in Exhibit 3 with self-attention) configured to generate, based in part on epistasis expressed by amino acid patterns spanning the target protein sequence and a plurality of non-target protein sequences aligned with the target protein sequence in a multiple sequence alignment,
   a second score-to-rank mapping that maps a second set of pathogenicity scores for the set of variants to a second set of score rankings;
a reassignment logic configured to reassign pathogenicity scores from the first set of pathogenicity scores to the set of variants based on the first and second score-to-rank mappings (as illustrated by the example in slide 26 of Exhibit 6); and
an output logic configured to generate a ranking of the set of variants based on the reassigned pathogenicity scores (as illustrated by the example in slide 26 of Exhibit 6 by 100% DeepSequence).

2. The system of clause 1, wherein the first score-to-rank mapping assigns a given variant in the set of variants a given pathogenicity score from the first set of pathogenicity scores.

3. The system of clause 2, wherein the second score-to-rank mapping assigns the given variant a given score ranking from the second set of score rankings.

4. The system of clause 3, wherein the first score-to-rank mapping assigns the given score ranking an another pathogenicity score from the first set of pathogenicity scores, wherein the another pathogenicity score is different from the given pathogenicity score.

5. The system of clause 4, wherein the reassignment logic is further configured to reassign the given variant the another pathogenicity score.

6. The system of clause 5, further configured to comprise a combination logic configured to combine the another pathogenicity score and the given pathogenicity score to generate a combined pathogenicity score.

7. The system of clause 6, wherein the reassignment logic is further configured to reassign the given variant the combined pathogenicity score.

8. The system of clause 6, wherein the combined pathogenicity score is an average of the another pathogenicity score and the given pathogenicity score (as illustrated by the example in slide 26 of Exhibit 6 by 50% DeepSequence).

9. The system of clause 8, wherein the combined pathogenicity score is a weighted average of the another pathogenicity score and the given pathogenicity score.

10. The system of clause 9, wherein weights used for the weighted average are preset and respectively specified for the first model and the second model.

11. The system of clause 10, wherein the weights correspond to respective rankings of the another pathogenicity score and the given pathogenicity score.

12. The system of clause 6, wherein the combined pathogenicity score is a sum of the another pathogenicity score and the given pathogenicity score.

13. The system of clause 12, wherein the combined pathogenicity score is a weighted sum of the another pathogenicity score and the given pathogenicity score.

14. The system of clause 13, wherein weights used for the weighted sum are preset and respectively specified for the first model and the second model.

15. The system of clause 14, wherein the weights correspond to respective rankings of the another pathogenicity score and the given pathogenicity score.

16. The system of clause 1, wherein the first model uses a first scale of pathogenicity scores to differentiate pathogenic variants from benign variants.

17. The system of clause 16, wherein the first scale ranges from 0 to 1.

18. The system of clause 1, wherein the second model uses a second scale of pathogenicity scores to differentiate pathogenic variants from benign variants.

19. The system of clause 18, wherein the second scale ranges from a maximum real number represented digitally (e.g., +∞) to a minimum real number represented digitally (e.g., -∞).

20. The system of clause 1, wherein the first model is further configured to generate the first set of pathogenicity scores based in part on three-dimensional (3D) structural information about the amino acids in the target protein sequence.

21. The system of clause 1, wherein the first model uses voxelized features as input.

22. The system of clause 1, wherein the first model is a site-independent model that factorizes single-position variations in a plurality of aligned sequences.

23. The system of clause 22, wherein the first model is a pairwise-interaction model that factorizes two-position variations in the plurality of aligned sequences.

24. The system of clause 23, wherein the second model is a non-linear latent variable model that posts hidden variables to jointly detect global patterns and local patterns of sequence variations across windows spanning multiple positions and multiple sequences in the plurality of aligned sequences.

25. The system of clause 24, wherein the second model is a protein language model.

26. The system of clause 25, wherein the second model is a Transformer.

27. The system of clause 24, wherein the second model is a variational autoencoder (VAE).

28. The system of clause 24, wherein the second model is a generative adversarial network (GAN).

29. The system of clause 1, wherein the plurality of non-target protein sequences are homologous with the target protein sequence.

30. The system of clause 1, wherein non-target protein sequences in the plurality of non-target protein sequences are homologous with the target protein sequence.

31. The system of clause 30, wherein the target protein sequence belongs to a first species.

32. The system of clause 31, wherein the first species is human.

33. The system of clause 30, wherein the non-target protein sequences belong to a first group of species that shares a genus with the first species.

34. The system of clause 33, wherein the genus is homo.

35. The system of clause 33, wherein the first group of species shares a family with the first species.

36. The system of clause 35, wherein the family is hominids.

37. The system of clause 33, wherein the first group of species shares an order with the first species.

38. The system of clause 37, wherein the order is primates.

39. The system of clause 30, wherein the non-target protein sequences belong to a second group of species that shares a class with the first species.

40. The system of clause 39, wherein the class is mammals.

41. The system of clause 39, wherein the second group of species shares a phylum with the first species.

42. The system of clause 41, wherein the phylum is chordates.

43. The system of clause 39, wherein the second group of species shares a kingdom with the first species.

44. The system of clause 43, wherein the kingdom is animals.

45. The system of clause 1, wherein the evolutionary conservation summary statistics are determined from evolutionary profiles.

46. The system of clause 45, wherein the evolutionary profiles include position-specific score matrices (PSSMs).

47. The system of clause 45, wherein the evolutionary profiles include position-specific frequency matrices (PSFMs).

48. The system of clause 1, wherein the second model is configured to capture higher-order epistatic interactions between mutations that span multiple positions and multiple sequences in the multiple sequence alignment.

49. The system of clause 48, wherein the higher-order epistatic interactions between the mutations form an undirected graph trajectory.

50. The system of clause 48, wherein the higher-order epistatic interactions include more than two-way epistatic interactions.

51. The system of clause 1, wherein the multiple sequence alignment aligns at least hundred sequences.

52. The system of clause 1, wherein the multiple sequence alignment aligns at least five hundred sequences.

53. The system of clause 1, wherein the epistasis is co-evolutionary epistasis.

54. The system of clause 1, wherein the amino acid patterns include at least three amino acids.

55. The system of clause 1, wherein the amino acid patterns are variant/mutation patterns.

56. A system, comprising:
a first model configured to generate, based in part on a target protein sequence,
   a first score-to-rank mapping that maps a first set of pathogenicity scores for a set of variants observed in the target protein sequence to a first set of score rankings;
a second model configured to generate, based in part on the target protein sequence and a plurality of non-target homologous protein sequences aligned with the target protein sequence in a multiple sequence alignment,
   a second score-to-rank mapping that maps a second set of pathogenicity scores for the set of variants to a second set of score rankings;
a reassignment logic configured to reassign pathogenicity scores from the first set of pathogenicity scores to the set of variants based on the first and second score-to-rank mappings; and
an output logic configured to generate a ranking of the set of variants based on the reassigned pathogenicity scores.

57. A system, comprising:
a first model configured to generate, based in part on position-specific evolutionary conservation statistics of amino acids in a target protein sequence,
   a first score-to-rank mapping that maps a first set of pathogenicity scores for a set of variants observed in the target protein sequence to a first set of score rankings;
a second model configured to generate, based in part on epistasis expressed by amino acid patterns spanning the target protein sequence and a plurality of non-target protein sequences aligned with the target protein sequence in a multiple sequence alignment,
   a second score-to-rank mapping that maps a second set of pathogenicity scores for the set of variants to a second set of score rankings;
a reassignment logic configured to reassign pathogenicity scores from the second set of pathogenicity scores to the set of variants based on the first and second score-to-rank mappings; and
an output logic configured to generate a ranking of the set of variants based on the reassigned pathogenicity scores.

58. A system, comprising:
a first model configured to generate, based in part on a target protein sequence,
   a first score-to-rank mapping that maps a first set of pathogenicity scores for a set of variants observed in the target protein sequence to a first set of score rankings;
a second model configured to generate, based in part on the target protein sequence and a plurality of non-target homologous protein sequences aligned with the target protein sequence in a multiple sequence alignment,
a second score-to-rank mapping that maps a second set of pathogenicity scores for the set of variants to a second set of score rankings;
a reassignment logic configured to reassign pathogenicity scores from the second set of pathogenicity scores to the set of variants based on the first and second score-to-rank mappings; and
an output logic configured to generate a ranking of the set of variants based on the reassigned pathogenicity scores.

59. A system, comprising:
a first model configured to generate, based in part on three-dimensional (3D) structural information about amino acids in a target protein sequence,
   a first score-to-rank mapping that maps a first set of pathogenicity scores for a set of variants observed in the target protein sequence to a first set of score rankings;
a second model configured to generate, based in part on the target protein sequence and a plurality of non-target homologous protein sequences aligned with the target protein sequence in a multiple sequence alignment,
a second score-to-rank mapping that maps a second set of pathogenicity scores for the set of variants to a second set of score rankings;
a reassignment logic configured to reassign pathogenicity scores from the first set of pathogenicity scores to the set of variants based on the first and second score-to-rank mappings; and
an output logic configured to generate a ranking of the set of variants based on the reassigned pathogenicity scores.

60. A system, comprising:
a first model configured to generate, based in part on three-dimensional (3D) structural information about amino acids in a target protein sequence,
a first score-to-rank mapping that maps a first set of pathogenicity scores for a set of variants observed in the target protein sequence to a first set of score rankings;
a second model configured to generate, based in part on the target protein sequence and a plurality of non-target homologous protein sequences aligned with the target protein sequence in a multiple sequence alignment,
a second score-to-rank mapping that maps a second set of pathogenicity scores for the set of variants to a second set of score rankings;
a reassignment logic configured to reassign pathogenicity scores from the second set of pathogenicity scores to the set of variants based on the first and second score-to-rank mappings; and
an output logic configured to generate a ranking of the set of variants based on the reassigned pathogenicity scores.

61. A system, comprising:
a first model configured to generate a first score-to-rank mapping that maps a first set of pathogenicity scores for a set of variants observed in a target protein sequence to a first set of score rankings;
a second model configured to generate a second score-to-rank mapping that maps a second set of pathogenicity scores for the set of variants to a second set of score rankings;
a reassignment logic configured to reassign pathogenicity scores from the second set of pathogenicity scores to the set of variants based on the first and second score-to-rank mappings; and
an output logic configured to generate a ranking of the set of variants based on the reassigned pathogenicity scores.

62. A system, comprising:
a first model configured to generate a first score-to-rank mapping that maps a first set of pathogenicity scores for a set of variants observed in a target protein sequence to a first set of score rankings, wherein pathogenicity scores in the first set of pathogenicity scores are scored on a first scale;
a second model configured to generate a second score-to-rank mapping that maps a second set of pathogenicity scores for the set of variants to a second set of score rankings, wherein pathogenicity scores in the second set of pathogenicity scores are scored on a second scale that is different from the first scale;
a rescaling logic configured to use the first and second score-to-rank mappings to rescale the pathogenicity scores in the second set of pathogenicity scores from the second scale to the first scale; and
an output logic configured to generate a ranking of the set of variants based on the rescaled pathogenicity scores.

### Clause Set 2

1. A system, comprising:
a first model (e.g., PrimateAI, PrimateAI 3D) configured to generate, based in part on evolutionary conservation summary statistics of amino acids in a reference target protein sequence, a first set of pathogenicity scores for a set of variants that mutate the reference target protein sequence to a set of alternate protein sequences, wherein the first set of pathogenicity scores has a first set of score rankings;
a second model (e.g., DeepSequence in Exhibit 7, Transformer-based models (e.g., the one shown in Exhibit 3 with self-attention) configured to generate, based in part on epistasis expressed by amino acid patterns spanning a multiple sequence alignment that aligns the reference target protein sequence to a plurality of non-target protein sequences, a second set of pathogenicity scores for the set of variants, wherein the second set of pathogenicity scores has a second set of score rankings;
a rank loss determination logic configured to determine a rank loss parameter based on a comparison of the first set of score rankings against the second set of score rankings;
a loss function reconfiguration logic configured to reconfigure a loss function based on the rank loss parameter; and
a training logic configured to use the reconfigured loss function to train the first model.

2. The system of clause 1, wherein the second model processes respective alternate protein sequences in the set of alternate protein sequences as respective inputs and generates respective pathogenicity scores in the second set of pathogenicity scores as respective outputs.

3. The system of clause 2, wherein the second model is pre-trained to process the multiple sequence alignment as an input and generate a reconstruction of the multiple sequence alignment as an output.

4. The system of clause 3, wherein the second model represents a reconstruction of a given alternate protein sequence as base-wise probability scores for each amino acid in the given alternate protein sequence.

5. The system of clause 4, wherein a joint probability determined from the base-wise probability scores is used as a pathogenicity score for a given variant that mutates the reference target protein sequence to the given alternate protein sequence.

6. The system of any of clauses 1-5, wherein respective coefficient and latent space configurations of the second model are pre-trained to process and reconstruct respective multiple sequence alignments that have respective reference target protein sequences as respective query sequences.

7. The system of clause 6, wherein the second model has a particular coefficient and latent space configuration corresponding to the reference target protein sequence.

8. The system of any of clauses 1-7, wherein the second model has one to twenty thousand coefficient and latent space configurations corresponding to one to twenty thousand reference protein sequences in human proteome.

9. The system of any of clauses 1-8, wherein the rank loss determination logic is further configured to determine the rank loss parameter (e.g., the rank losses and variants discussed in Exhibit 12) based on a combination of the first set of score rankings and the second set of score rankings.

10. The system of clause 9, wherein the combination is a weighted combination.

11. The system of clause 10, wherein weights used to generate the weighted combination are preset.

12. The system of clause 11, wherein the weights are differentiable and learned in a re-ranking layer that is trained as part of the training of the first model.

13. The system of any of clauses 1-12, wherein the second model is a variational autoencoder (VAE).

14. The system of any of clauses 1-13, wherein the second model is a generative adversarial network (GAN).

15. The system of any of clauses 1-14, further configured to comprise:
a third model e.g., DeepSequence in Exhibit 7, Transformer-based models (e.g., the one shown in Exhibit 3 with self-attention) configured to generate, based in part on the epistasis expressed by the amino acid patterns spanning the multiple sequence alignment, a third set of pathogenicity scores for the set of variants, wherein the third set of pathogenicity scores has a third set of score rankings;
the rank loss determination logic further configured to determine the rank loss parameter based on a comparison of the first set of score rankings, the second set of score rankings, and the third set of score rankings;
the loss function reconfiguration logic further configured to reconfigure the loss function based on the rank loss parameter; and
the training logic further configured to use the reconfigured loss function to train the first model.

16. The system of any of clauses 1-15, wherein the third model is a Transformer-based model.

17. The system of clause 15, wherein the rank loss determination logic is further configured to determine the rank loss parameter based on a combination of the first set of score rankings, the second set of score rankings, and the third set of score rankings.

18. The system of clause 17, wherein the combination is a weighted combination.

19. The system of clause 18, wherein weights used to generate the weighted combination are preset.

20. The system of clause 19, wherein the weights are differentiable and learned as part of the training of the first model.

21. The system of clause 20, wherein the weights are differentiable and learned in stacked re-ranking layers that are trained as part of the training of the first model using activation functions that generate non-linear combinations of the first set of score rankings, the second set of score rankings, and the third set of score rankings.

22. The system of any of clauses 1-21, further configured to comprise:
a fourth model (e.g., JigsawAI) configured to generate, based in part on masked representations of the evolutionary conservation summary statistics, a fourth set of pathogenicity scores for the set of variants, wherein the masked representations mask evolutionary conservation summary statistic data about at least one amino acid in the alternate protein sequences, and wherein the fourth set of pathogenicity scores has a fourth set of score rankings;
the rank loss determination logic further configured to determine the rank loss parameter based on a comparison of the first set of score rankings, the second set of score rankings, and the fourth set of score rankings;
the loss function reconfiguration logic further configured to reconfigure the loss function based on the rank loss parameter; and
the training logic further configured to use the reconfigured loss function to train the first model.

23. The system of any of clauses 1-22, further configured to comprise:
the rank loss determination logic further configured to determine the rank loss parameter based on a comparison of the first set of score rankings, the second set of score rankings, the fourth set of score rankings, and the fourth set of score rankings;
the loss function reconfiguration logic further configured to reconfigure the loss function based on the rank loss parameter; and
the training logic further configured to use the reconfigured loss function to train the first model.

24. The system of any of clauses 1-23, wherein the training logic is further configured to use the reconfigured loss function to train the fourth model.

25. The system of any of clauses 1-24, further configured to comprise:
the loss function reconfiguration logic further configured to reconfigure, based on the rank loss parameter, a first loss function for the first model and a fourth loss function for the fourth model; and
the training logic further configured to use the reconfigured first function to train the first model, and to use the reconfigured fourth function to train the fourth model.

26. The system of any of clauses 1-25, wherein the first model is further configured to generate, based in part on three-dimensional (3D) structural representations of amino acids in the reference target protein sequence, the first set of pathogenicity scores.

27. The system of any of clauses 1-26, wherein the first model is further configured to generate, based in part on the reference target protein sequence, the first set of pathogenicity scores.

28. The system of any of clauses 1-27, wherein the first model is further configured to generate, based in part on the alternate protein sequences, the first set of pathogenicity scores.

29. The system of any of clauses 1-28, wherein the fourth model is further configured to generate, based in part on masked representations of the 3D structural representations of the amino acids in the reference target protein sequence, the fourth set of pathogenicity scores, wherein the masked representations of the 3D structural representations mask 3D structural data about at least one amino acid in the reference target protein sequence.

30. The system of any of clauses 1-29, wherein the fourth model is further configured to generate, based in part on a masked representation of the reference target protein sequence, the fourth set of pathogenicity scores, wherein the masked representation masks at least one amino acid in the reference target protein sequence.

31. The system of any of clauses 1-30, wherein the fourth model is further configured to generate, based in part on masked representations of the alternate protein sequences, the fourth set of pathogenicity scores, wherein the masked representations of the alternate protein sequences mask at least one amino acid in the reference target protein sequence.

32. The system of any of clauses 1-31, wherein the evolutionary conservation summary statistics are determined from evolutionary profiles.

33. The system of any of clauses 1-32, wherein the evolutionary profiles include position-specific score matrices (PSSMs).

34. The system any of clauses 1-33, wherein the evolutionary profiles include position-specific frequency matrices (PSFMs).

35. The system any of clauses 1-34, wherein the reference target protein sequence is a subsequence in a region in the reference target protein sequence.

36. The system any of clauses 1-35, wherein the alternate protein sequences are sub-sequences in regions in the alternate protein sequences.

What We claim is:

1. A system, comprising:
a first model configured to generate, based in part on evolutionary conservation summary statistics of amino acids in a target protein sequence,
a first score-to-rank mapping that maps a first set of pathogenicity scores for a set of variants observed in the target protein sequence to a first set of score rankings;
a second model configured to generate, based in part on epistasis expressed by amino acid patterns spanning the target protein sequence and a plurality of non-target protein sequences aligned with the target protein sequence in a multiple sequence alignment,
a second score-to-rank mapping that maps a second set of pathogenicity scores for the set of variants to a second set of score rankings;
a reassignment logic configured to reassign pathogenicity scores from the first set of pathogenicity scores to the set of variants based on the first and second score-to-rank mappings; and
an output logic configured to generate a ranking of the set of variants based on the reassigned pathogenicity scores.

2. The system of claim 1, wherein the first score-to-rank mapping assigns a given variant in the set of variants a given pathogenicity score from the first set of pathogenicity scores.

3. The system of claim 2, wherein the second score-to-rank mapping assigns the given variant a given score ranking from the second set of score rankings.

4. The system of claim 3, wherein the first score-to-rank mapping assigns the given score ranking an another pathogenicity score from the first set of pathogenicity scores, wherein the another pathogenicity score is different from the given pathogenicity score.

5. The system of claim 4, wherein the reassignment logic is further configured to reassign the given variant the another pathogenicity score.

6. The system of claim 5, further configured to comprise a combination logic configured to combine the another pathogenicity score and the given pathogenicity score to generate a combined pathogenicity score.

7. The system of claim 6, wherein the reassignment logic is further configured to reassign the given variant the combined pathogenicity score.

8. The system of claim 6, wherein the combined pathogenicity score is an average of the another pathogenicity score and the given pathogenicity score.

9. The system of claim 8, wherein the combined pathogenicity score is a weighted average of the another pathogenicity score and the given pathogenicity score.

10. The system of claim 9, wherein weights used for the weighted average are preset and respectively specified for the first model and the second model.

11. The system of claim 10, wherein the weights correspond to respective rankings of the another pathogenicity score and the given pathogenicity score.

12. The system of claim 6, wherein the combined pathogenicity score is a sum of the another pathogenicity score and the given pathogenicity score.

13. The system of claim 12, wherein the combined pathogenicity score is a weighted sum of the another pathogenicity score and the given pathogenicity score.

14. The system of claim 13, wherein weights used for the weighted sum are preset and respectively specified for the first model and the second model.

15. The system of claim 14, wherein the weights correspond to respective rankings of the another pathogenicity score and the given pathogenicity score.

16. The system of claim 1, wherein the first model uses a first scale of pathogenicity scores to differentiate pathogenic variants from benign variants.

17. The system of claim 16, wherein the first scale ranges from 0 to 1.

18. The system of claim 1, wherein the second model uses a second scale of pathogenicity scores to differentiate pathogenic variants from benign variants.

19. The system of claim 18, wherein the second scale ranges from wherein the second scale ranges from a maximum real number represented digitally to a minimum real number represented digitally.

20. The system of claim 1, wherein the first model is further configured to generate the first set of pathogenicity scores based in part on three-dimensional (3D) structural information about the amino acids in the target protein sequence.

21. The system of claim 1, wherein the first model uses voxelized features as input.

22. The system of claim 1, wherein the first model is a site-independent model that factorizes single-position variations in a plurality of aligned sequences.

23. The system of claim 22, wherein the first model is a pairwise-interaction model that factorizes two-position variations in the plurality of aligned sequences.

24. The system of claim 23, wherein the second model is a non-linear latent variable model that posts hidden variables to jointly detect global patterns and local patterns of sequence variations across windows spanning multiple positions and multiple sequences in the plurality of aligned sequences.
